(19) United States
(12) Patent Application Publication
(10) Pub. No.: US 2025/0066823 A1
(43) Pub. Date: Feb. 27, 2025

(54) METHODS FOR ENGINEERING OUTER MEMBRANE VESICLE PRODUCTION AND CARGO PACKAGING IN PSEUDOMONAS PUTIDA

(71) Applicant: Alliance for Sustainable Energy, LLC, Golden, CO (US)

(72) Inventors: Rebecca Ann WILKES, Lakewood, CO (US); Allison Jean ZIMONT WERNER, Denver, CO (US); Tarryn E. MILLER, Wheat Ridge, CO (US); Gregg Tyler BECKHAM, Golden, CO (US)

(21) Appl. No.: 18/787,311

(22) Filed: Jul. 29, 2024

Related U.S. Application Data
(60) Provisional application No. 63/516,377, filed on Jul. 28, 2023.
(60) Provisional application No. 63/581,191, filed on Sep. 7, 2023.

(51) Int. Cl. C12P 7/44; C12N 1/20; C12N 9/02; C12R 1/39; C12R 1/40

(52) U.S. Cl. CPC C12P 7/44; C12N 1/20; C12N 9/0069; C12R 2001/39; C12R 2001/40; C12Y 113/11002

(57) ABSTRACT

Disclosed herein are methods, compositions and systems useful for genetically engineering subcellular compartments such as OMVs for synthetic biology applications. In an embodiment, genetically engineered bacteria use OMVs to secrete compounds or proteins of interest extracellularly where the compounds or proteins of interest can be isolated from the growth media.

### CROSS-REFERENCE TO RELATED APPLICATIONS

This application claims priority under 35 U.S.C. § 119 to U.S. provisional patent application No. 63/516,377 filed on Jul. 28, 2023 and U.S. provisional patent application No. 63/581,191 filed on Sep. 7, 2023, the contents of which are hereby incorporated in their entirety.

### CONTRACTUAL ORIGIN

This invention was made with government support under Contract No. DE-AC36-08GO28308 awarded by the Department of Energy. The government has certain rights in the invention.

### SEQUENCE LISTING

The instant application contains a Sequence Listing which has been submitted electronically and is hereby incorporated by reference in its entirety. The XML copy as filed herewith was originally created on Oct. 18, 2024 is named NREL 23-96.xml and is 54 kilobytes in size.

### BACKGROUND

Outer membrane vesicles (OMVs) are produced by gram-negative bacteria and represent a currently untapped resource for bioprocess engineering. Hydrophobic products without designated secretion mechanisms, such as carotenoids, curcuminoids, and other natural products can accumulate in the cell membrane and require cell lysis to extract the products. Thus, engineering increased vesiculation has potential to act as a secretion mechanism for these specialty chemicals. Further, genetic tools to target specific enzymes to OMVs, would enable OMVs to be utilized as biocatalysts with coordinated enzymatic reactions. This directed spatial organization of enzymatic reactions between cell and OMV also has the potential to increase enzyme stability over free enzymes and for improving the detoxification of chemicals extracellularly prior to interference with intracellular machinery. Therefore, there is a need for innovative methods, systems, and organisms that can convert aromatic compounds derived from waste and renewable resources to commodity and specialized chemicals.

### SUMMARY

In an aspect, disclosed herein is a genetically modified Pseudomonas sp. comprising at least one deletion of an endogenous gene, wherein the one or more deletion results in an increase in the production of outer membrane vesicles (OMVs) relative to the wild-type Pseudomonas sp. In an embodiment, the endogenous gene is selected from the group consisting of oprF, and oprI. In an embodiment, the Pseudomonas sp. is selected from the group consisting of P. putida, P. fluorescens, and P. stutzeri. In an embodiment, the P. putida is P. putida KT2440.

In an aspect, disclosed herein is a genetically modified Pseudomonas sp. comprising at least one deletion of an endogenous gene, wherein: the one or more deletion results in an increase in the production of outer membrane vesicles (OMVs) relative to the wild-type Pseudomonas sp.; and wherein the genetically modified Pseudomonas sp. further comprises at least one exogenous gene encoding an enzyme; and wherein the expressed enzyme encoded by the at least one exogenous gene encoding an enzyme is connected to an outer membrane protein that is incorporated into the membrane of an outer membrane vesicle; and wherein the enzyme is connected to the outer membrane protein through a linker. In an embodiment, the expressed enzyme encoded by the at least one exogenous gene is connected to the endogenous outer membrane protein by a protein linker. In an embodiment, the expressed enzyme encoded by the at least one exogenous gene is tagged with a vesicle nucleating peptide. In an embodiment, the expressed enzyme encoded by the at least one exogenous gene is tagged with a vesicle nucleating peptide having a sequence selected from the group consisting of SEQ ID NO: 5, SEQ ID NO: 6 and SEQ ID NO: 7.

In an aspect, disclosed herein is a system for the production and isolation of a compound of interest comprising a genetically modified Pseudomonas sp. comprising at least one deletion of an endogenous gene, wherein the one or more deletion results in an increase in the production of outer membrane vesicles (OMVs) relative to the wild-type Pseudomonas sp.; and wherein the genetically modified Pseudomonas sp. further comprises at least one exogenous gene encoding an enzyme; and wherein the expressed enzyme encoded by the at least one exogenous gene encoding an enzyme is connected to an outer membrane protein that is incorporated into the membrane of an outer membrane vesicle; and wherein the expressed enzyme is connected to the outer membrane protein through a linker; and wherein the expressed enzyme encoded by the at least one exogenous gene is contacted with a substrate; and wherein a product of a reaction catalyzed by the expressed enzyme encoded by the at least one exogenous gene is isolated; and wherein the product of the reaction catalyzed by the expressed enzyme is the compound of interest. In an embodiment, the expressed enzyme encoded by the at least one exogenous gene is XylE; and the substrate is catechol and the product is 2-hydroxymuconic semialdehyde. In an embodiment, the expressed enzyme encoded by the at least one exogenous gene is isolated. In an embodiment, the outer membrane protein is endogenous. In an embodiment, the outer membrane protein is selected from the group consisting of OmpA (PP_1122) and EstP. In an embodiment, the outer membrane protein is OmpA (PP_1122) and wherein the expressed enzyme encoded by the at least one exogenous gene is on the inside of the outer membrane vesicle. In an embodiment, the outer membrane protein is EstP and wherein the expressed enzyme encoded by the at least one exogenous gene is on the outside of the outer membrane vesicle. In an embodiment, the outer membrane protein is exogenous. In an embodiment, the outer membrane protein is selected from the group consisting of OmpA from Escherichia coli or INP from Pseudomonas syringae. In an embodiment, the outer membrane protein is OmpA from Escherichia coli and wherein the expressed enzyme encoded by the at least one exogenous gene is on the inside of the outer membrane vesicle. In an embodiment, the outer membrane protein is INP from Pseudomonas syringae and wherein the expressed enzyme encoded by the at least one exogenous gene is on the outside of the outer membrane vesicle. In an embodiment, the expressed enzyme encoded by the at least one exogenous gene is tagged with a vesicle nucleating peptide. In an embodiment, the expressed enzyme encoded by the at least one exogenous gene is tagged with a vesicle nucleating peptide having a sequence selected from the group consisting of SEQ ID NO: 5, SEQ ID NO: 6 and SEQ ID NO: 7. In an embodiment, the genetically modified Pseudomonas sp. is selected from the group consisting of P. putida, P. fluorescens, and P. stutzeri.

### BRIEF DESCRIPTION OF THE DRAWINGS

Some embodiments are illustrated in referenced figures of the drawings. It is intended that the embodiments and figures disclosed herein are to be considered illustrative rather than limiting.

FIGS. 1A and 1B depict two genetic knockouts that initiate hypervesiculation relative to WT during growth on glucose alone. FIG. 1A depicts nanoparticle tracking analysis that was used to enumerate the OMVs in the knockout strains in comparison to WT and WT exposed to 50 μM Pseudomonas quinolone signal (2-heptyl-3-hydroxy-4 (1H)-quinolone; PQS) as a positive control for hypervesiculation. Significant differences relative to the wild-type strain are illustrated with the p-values from a one-tailed T-test. FIG. 1B depicts nanoparticle tracking analysis of the size distribution of the OMVs in the different strains.

FIG. 2 depicts a bicinchoninic acid (BCA) assay on the OMV fraction collected for each strain grown on 20 mM glucose was used to quantify the membrane protein amount as a proxy for assessing hypervesiculation phenotype in knockout strains.

FIGS. 3A and 3B depict an embodiment wherein the hypervesiculation phenotype is independent of the substrates provided to support growth. Both ΔoprF and ΔoprI increase vesicle production relative to the wild-type strain when grown on 20 mM glucose, 12.5 mM p-coumarate, and 12.5 mM ferulate. FIG. 3A depicts a nanoparticle tracking analysis that was used to enumerate the OMVs in the knockout strains in comparison to WT. Significant differences relative to the wild-type strain are illustrated with the p-values from a one-tailed T-test. FIG. 3B depicts a nanoparticle tracking analysis of the size distribution of the OMVs in the different strains.

FIGS. 4A and 4B depict the design of a spytag-spycatcher (ST-SC) display system in P. putida KT2440. FIG. 4A depicts a schematic of targeting the cargo enzyme into the periplasm for internal display inside the OMV versus targeting of cargo enzyme outside of the cell for external display on the OMV. FIG. 4B depicts an overview of the genetic constructs used to create strains with ST-SC internal or external display of an example of a cargo protein, XylE.

FIGS. 5A-5C depict four strains (RW90, RW91, RW92, and RW93) with a SC-anchor that directed XylE activity extracellularly relative to the control strain RW87. FIG. 5A depicts a propidium iodide assay that was used to assess the membrane permeability of the strains. The membrane permeability was not increased in the engineered strains indicating that the proteins were not just passing through a permeabilized membrane. FIG. 5B depicts the dynamics of product formation (2-hydroxymuconic semialdehyde) measured at 375 nm after the addition of 0.25 mM catechol to the 10× concentrated supernatant. The control of XylE-ST, without an SC-anchor to target the enzyme to the OMVs, did not show activity in the supernatant. FIG. 5C depicts calculated initial rates relative to the total protein concentration in the supernatant and maximum product (HMS) concentration achieved for each strain after 200 min.

FIG. 6 depicts fluorescence measurements of strains, both cells and supernatant, expressing (mNeongreen) mNG with various peptide tags. In an embodiment, peptide tags such as vesicle nucleating peptides (vNPs) vNP and vNP15 are used to increase the expression of mNG in the supernatant compared to the non-tagged mNG. The supernatant includes OMVs, indicating that different vNP tags change the relative fraction of mNG signal (e.g. in the supernatant vs. the whole washed cells). Cultures were in exponential phase in (N=2), cells were washed and normalized to OD600 nm in fresh M9 media.

FIG. 7 depicts a dot blot of three strains using anti-His antibodies to demonstrate that by using the vNP, mNeongreen (mNG) can be targeted into the outer membrane vesicle fraction and into the vesicle free secretome (VFS) of P. putida. The image shows the results of an experiment where these bacterial strains were grown and the cultures were then fractionated into whole cells, purified OMVs, and the vesicle free secretome. Each spot was normalized by total protein concentration. The brightness of a spot indicates the presence and level of expression (comparatively) of a His-tagged mNG in a particular strain or fraction. In TM27, mNG-His6 is present in both the vesicle free secretome and the purified OMVs.

FIGS. 8A and 8B depict lipid concentration and OMV mNG fluorescence. FIG. 8A depicts Nile red fluorescence of OMVs purified from two strains, TM26 and TM27. TM27 shows an increase in lipid concentration over time. Nile red interacts with lipids and is used as a proxy measurement for OMVs. FIG. 8B depicts relative fluorescence of mNG in OMVs purified from TM27 and TM26. TM27 has an increased fluorescence signal compared to TM26, indicating that vNP can target mNG into the OMVs of P. putida.

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Manipulating OMV biogenesis in bacteria allows for the use of OMVs as tools in synthetic biology and biotechnology. Successfully triggering OMV formation and targeting specific enzymes/proteins to locations in the OMV allows for the creation and modification of OMVs in a predictable and highly controlled fashion.

Disclosed herein are methods, compositions and systems useful for genetically engineering subcellular compartments such as OMVs for synthetic biology applications. In an embodiment, genetically engineered bacteria use OMVs to secrete compounds or proteins of interest extracellularly where the compounds or proteins of interest can be isolated from the growth media.

In an embodiment, disclosed herein are novel bacteria, e.g., P. putida, that are engineered to: 1) use genetic mechanisms to induce greater vesicle formation; and 2) to genetically target distinct enzymes either internal or external to OMVs. As described herein, four gene deletions were found that increase OMV production during growth on glucose in P. putida KT2440. Extraction of OMVs during exponential growth for P. putida ΔoprF and P. putida and ΔoprI exhibited higher particle counts per gCDW, representing greater production of OMVs, compared to the parent strain P. putida KT2440. Additionally, to target specific enzymes into the OMVs produced by P. putida, spytag-spycatcher (ST-SC) technology was used to engineer four protein anchors with spycatcher003 and a proof-of-concept enzyme with spytag003.

In an embodiment, P. putida ΔoprF, and P. putida ΔoprI initiated hypervesiculation relative to wild type (WT) during growth on glucose alone. As disclosed herein, nanoparticle tracking analysis was used to count and measure the OMVs in P. putida ΔoprF and P. putida ΔoprI in comparison to WT and WT exposed to 50 μM Pseudomonas quinolone signal (2-heptyl-3-hydroxy-4 (1H)-quinolone; PQS) as a positive control for hypervesiculation.

In an embodiment, the hypervesiculation phenotype is independent of the substrates provided to support growth. As an example, both ΔoprF and ΔoprI increase vesicle production relative to the wild-type strain when grown on 20 mM glucose, 12.5 mM p-coumarate, and 12.5 mM ferulate.

In an embodiment, a bicinchoninic acid (BCA) assay was performed on the OMV fraction collected for each knockout strain grown on 20 mM glucose. This assay was used to quantify the membrane protein amount as a proxy for assessing hypervesiculation phenotype in knockout strains.

Pseudomonas putida KT2440 was engineered to produce two new strains that were found to increase vesicle production by knocking out proteins found in the outer membrane of the cell (see Table 1). All gene deletions were conducted using pK18sB backbone with 1000 bp homology regions and sequenced confirmed before utilization.

Table 1 lists strains identified to have increased vesiculation relative to wildtype.

TABLE 1

| Strains | Genotype | Construction details | Protein |
|---|---|---|---|
| RW29 | P. putida KT2440 ΔPP_2089 | pRW007 was | Outer membrane porin F |
| | (ΔoprF) | transformed into | |
| | | KT2440. Deletion of | |
| | | oprF (PP_2089) was | |
| | | confirmed by colony | |
| | | PCR with oRW037 | |
| | | and oRW038 | |
| | | (Tm = 68° C., 2.3 kB) | |
| | | followed by Sanger | |
| | | sequencing | |
| RW30 | P. putida KT2440 ΔPP_2322 | pRW008 was | major outer membrane |
| | (ΔoprI) | transformed into | lipoprotein |
| | | KT2440. Deletion of | |
| | | oprI (PP_2322) was | |
| | | confirmed by colony | |
| | | PCR with oRW041 | |
| | | and oRW042 | |
| | | (Tm = 67° C., 2.3 kB) | |
| | | followed by Sanger | |
| | | sequencing | |

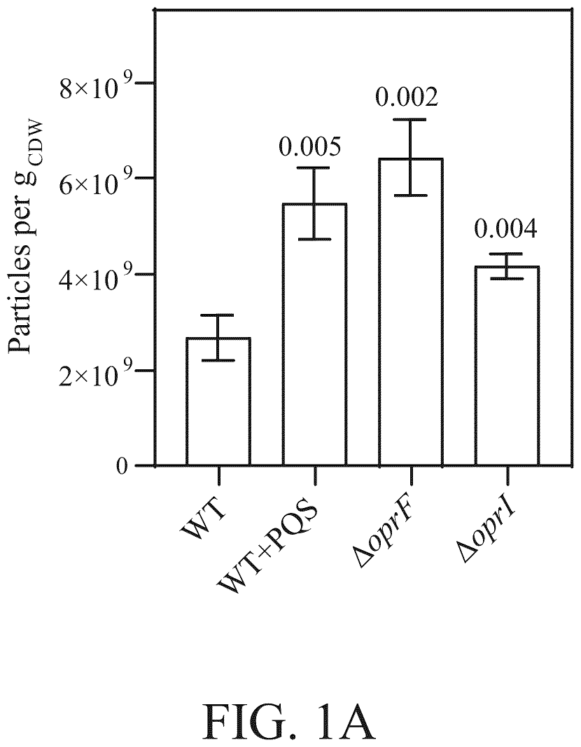

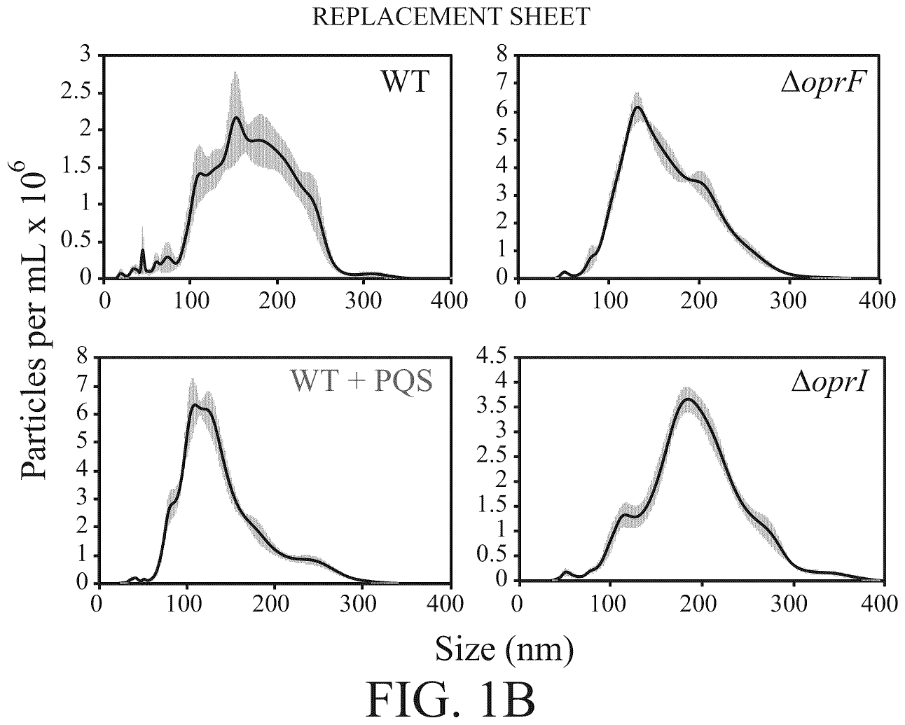

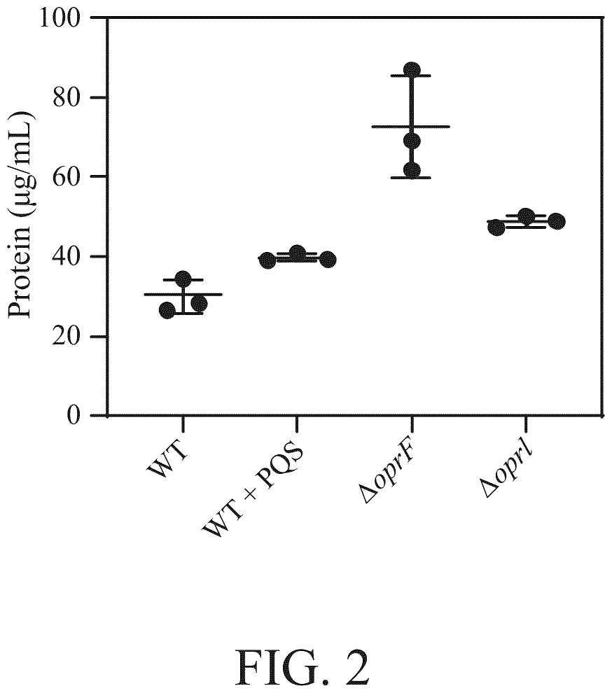

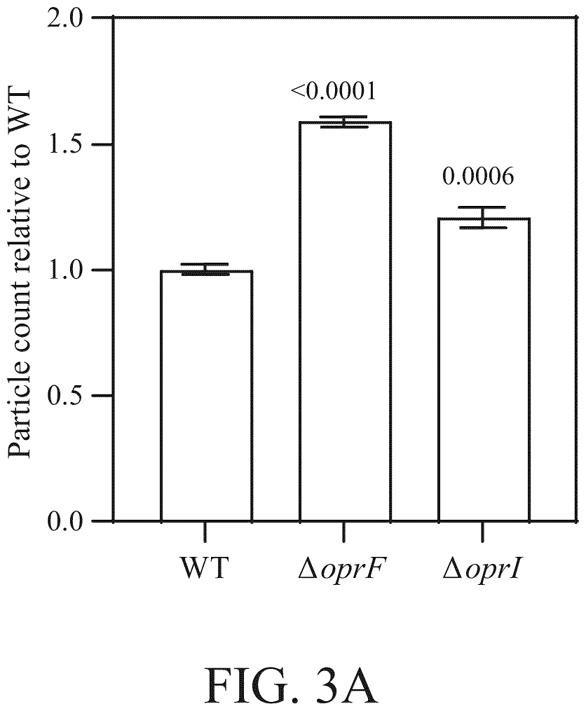

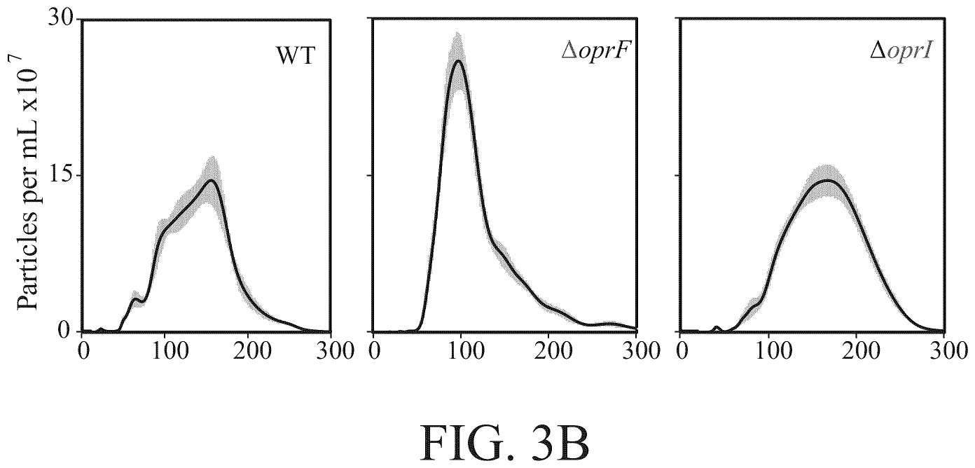

The strain with the greatest vesiculation (4-fold higher particle counts than WT) was RW29 (P. putida KT2440 ΔoprF) (see FIG. 1A). However, the hypervesiculation phenotype of ΔoprF was paired with reduced membrane integrity of the cell, a longer lag in growth, and diminished tolerance to the aromatic compounds coumarate and ferulate. Thus, this high production of OMVs impaired the overall cell performance. A more modest increase in vesicle formation was found for ΔoprI, which had a1.5-fold increased particle counts relative to WT (see FIG. 1A). These strains did not have impaired membrane integrity, growth phenotype, or tolerance to aromatic compounds. Overall, deletion of outer membrane porins or lipoproteins in P. putida KT2440 was an effective genetic tool to selectively increase the production of OMVs with or without interfering with the native cell growth phenotype.

Table 2 lists oligonucleotides used herein.

TABLE 2

| Name | Sequence (5′->3′) | Purpose |
|---|---|---|
| oRW037 | ATCGGCCTGGAATATTCGG | Colony PCR |
| | C (SEQ ID NO: 1) | of pRW007 |
| oRW038 | GACCGGACACTACCCGTAC | integration |
| | (SEQ ID NO: 2) | |
| oRW041 | GCTTGCAACGTGCCAATGC | Colony PCR |
| | (SEQ ID NO: 3) | of pRW008 |
| oRW042 | GGCCAACATCATGGTCGAC | integration |
| | (SEQ ID NO: 4) | |

Table 3 describes plasmids used herein.

TABLE 3

| Name | Description | Construction details |
|---|---|---|
| pRW007 | pK18sB-based | 1 kb homology regions upstream and |
| | plasmid for deletion of | downstream of PP_2089 (oprF) were |
| | PP_2089 (oprF) in | designed. An XbaI site was inserted |
| | P. putida KT2440- | between the two homology regions, |
| | derived strains | and the insert was cloned into the |
| | | pK18sb backbone at the EcoRI and |
| | | HindIII sites. The plasmid was |
| | | synthesized and sequence-verified by |
| | | Twist Biosciences. |
| pRW008 | pK18sB-based | 1 kb homology regions upstream and |
| | plasmid for deletion of | downstream of PP_2322 (oprI) were |
| | PP_2322 (oprI) in | designed. An XbaI site was inserted |
| | P. putida KT2440- | between the two homology regions, |
| | derived strains | and the insert was cloned into the |
| | | pK18sb backbone at the EcoRI and |
| | | HindIII sites. The plasmid was |
| | | synthesized and sequence-verified by |
| | | Twist Biosciences. |

In another embodiment, two native outer membrane proteins were chosen as anchors with fusion to the spycatcher003 sequence (PP_1122 and estP) and integrated into the genome of P. putida KT2440. The other two anchors fused to the spycatcher003 (ompA from Escherichia coli and inp from P. syringae) and were expressed on a pBTL-2 plasmid with arabinose induction and kanamycin resistance. To test the efficacy of this ST-SC system of outer membrane anchors to target enzymes into the OMVs, the enzyme XylE from P. putida mt-2 was fused with spytag003 and integrated into the genome of strains containing the anchors.

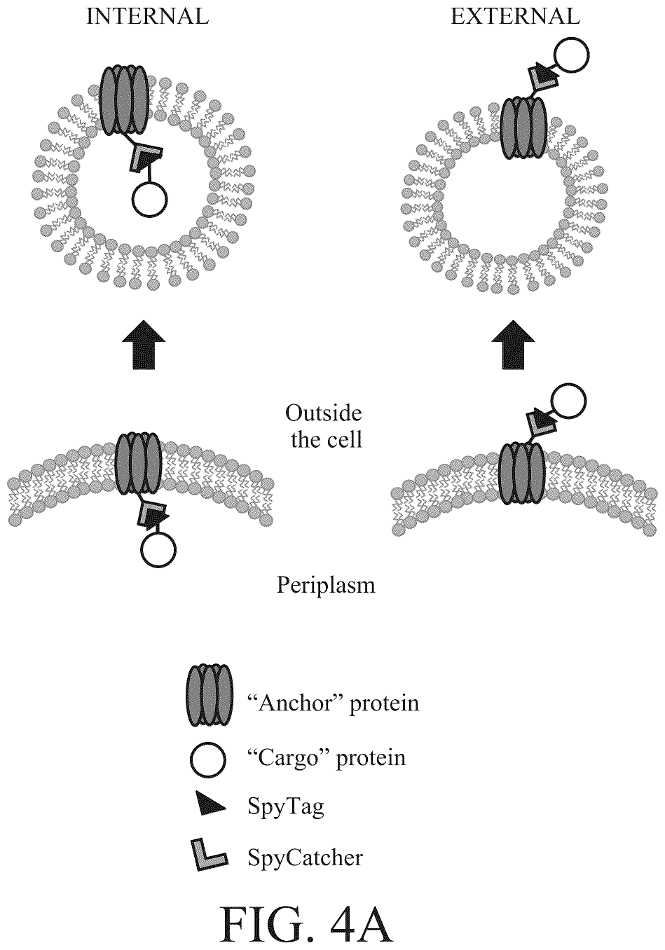

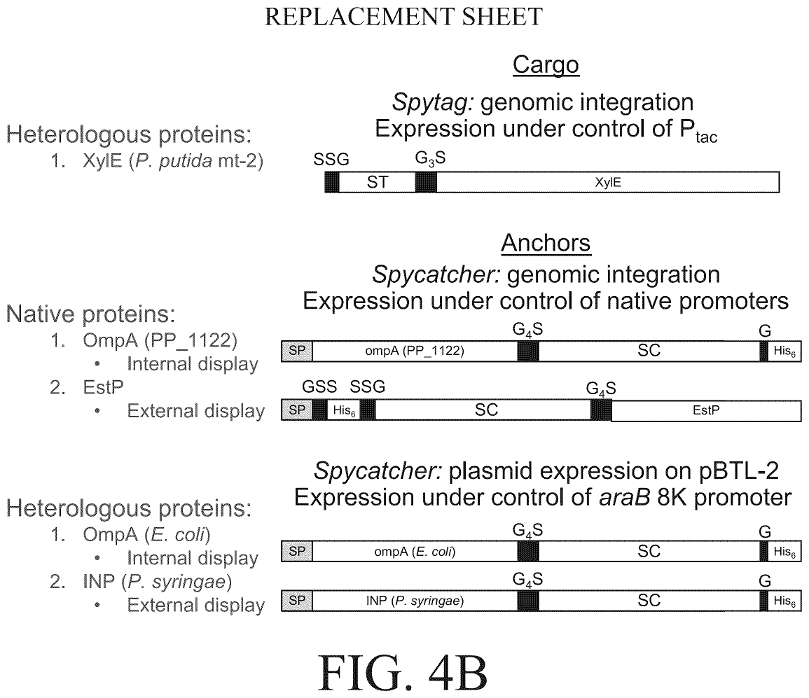

FIGS. 4A and 4B illustrate the design of a spytag-spycatcher (ST-SC) display system in P. putida KT2440. FIG. 4A is a schematic representation of targeting the cargo enzyme into the periplasm for internal display inside the OMV versus targeting of cargo enzyme outside of the cell for external display on the OMV. FIG. 4B depicts an overview of the genetic constructs used to create strains with ST-SC internal or external display of the cargo protein XylE. In an embodiment, the exemplary cargo enzyme XylE was fused to spytag003 and integrated into the genome with constitutive expression using Ptac. The four anchors tested here were individually incorporated into the base strain containing XylE-ST (RW87). Two native outer membrane proteins were used as anchors (PP_1122; ompA like protein) and EstP. For these proteins, the linker regions, spycatcher003, and a His-tag were integrated into the genome using homologous recombination. The native RBS and promoters were left intact. For the heterologous protein anchors from E. coli and P. syringae, the expressed outer membrane proteins were His-tagged, linked to spycatcher003 and their genetic sequences were encoded on the pBTL-2 plasmid under the control of an araB 8K promoter. Accordingly, expression of anchor proteins was induced with 1% wt/v arabinose.

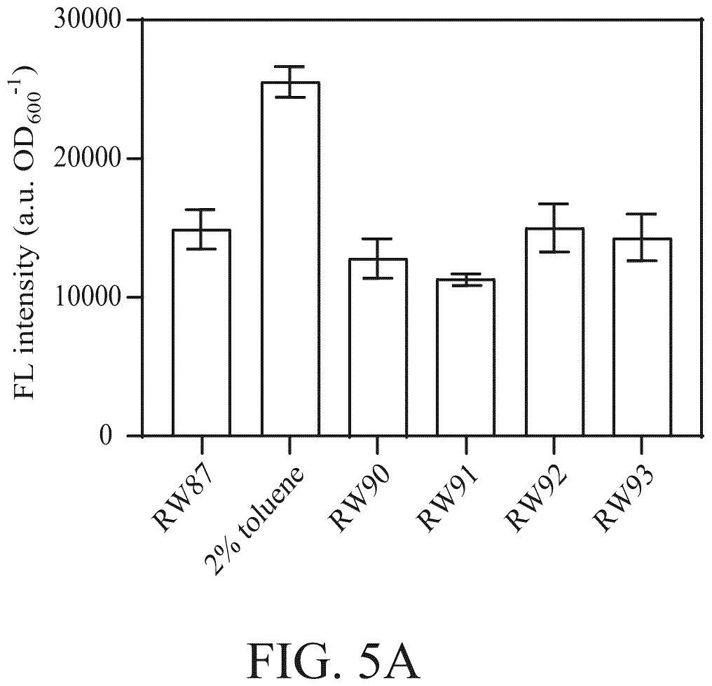

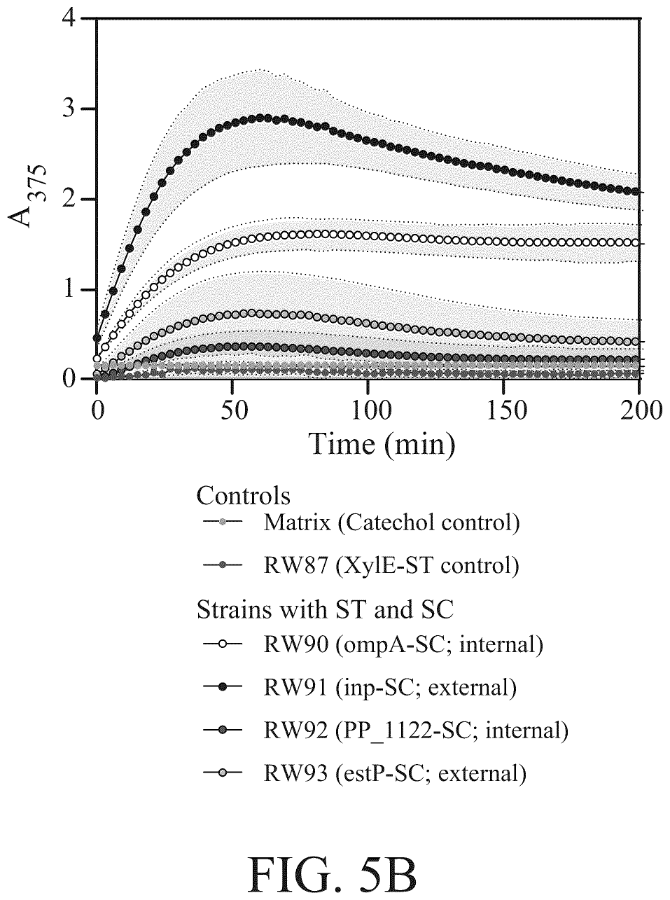

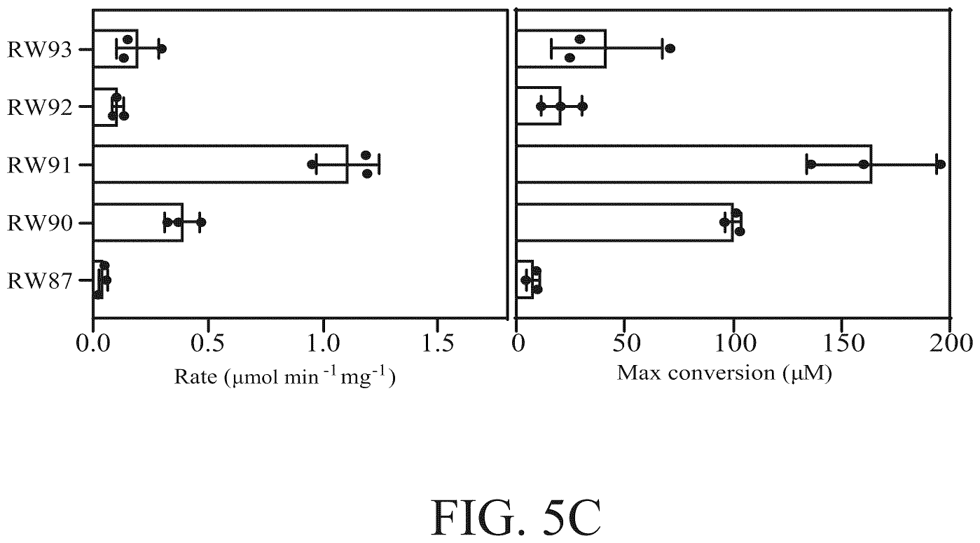

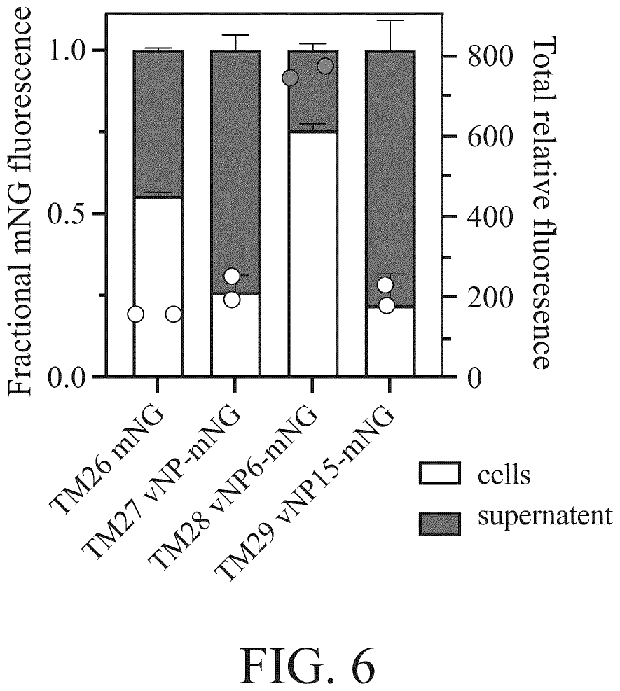

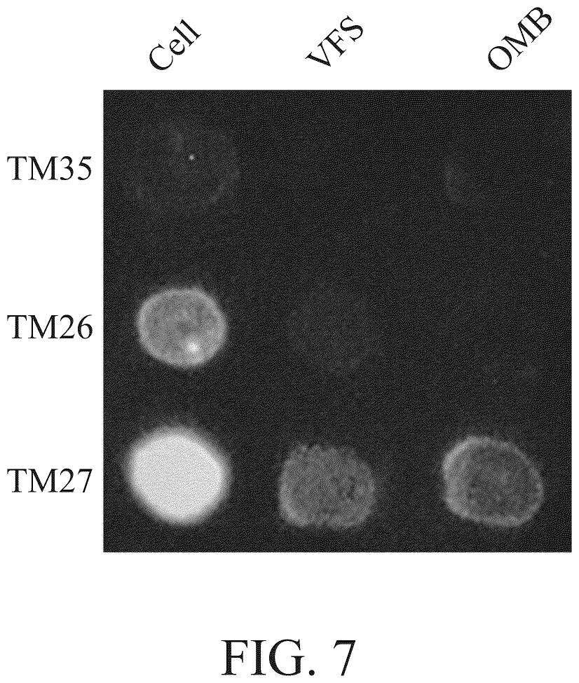

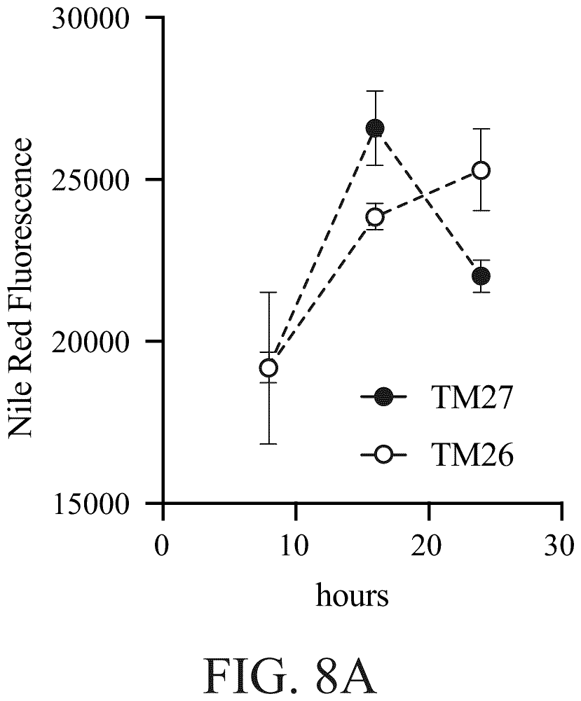

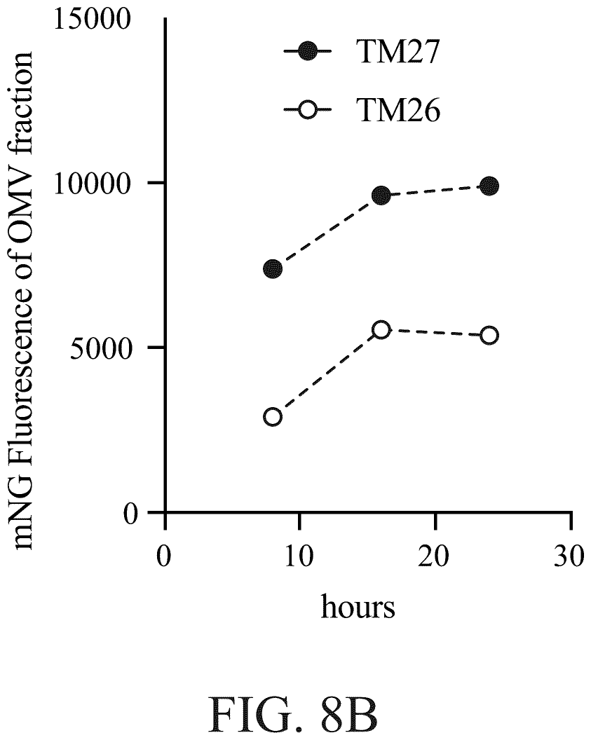

FIGS. 5A, 5B, and 5C depict data from experiments that shows that all four strains containing a spycatcher anchor that were combined with the spytag cargo (XylE) had enzymatic activity localized in the supernatant. The control of XylE-ST without an anchor to localize the enzyme to the OMVs did not show activity in the supernatant. Cells were grown for 24 h on LB broth, pelleted by centrifugation, and the supernatant was collected and concentrated using 30 kDa molecular weight cut-off filters (MWCO). The formation of 2-hydroxymuconic semialdehyde was measured at 375 nm after the addition of 0.25 mM catechol to the 10× concentrated supernatant.

Table 4 lists strains used herein in spytag-spycatcher targeting of enzymes into the OMVs.

TABLE 4

| Strains | Genotype | Details |
|---|---|---|
| RW87 | P. putida KT2440 ΔcatA2 | Parent strain with cargo protein (XylE) fused to |
| | ΔcatRBCA::Ptac:XylE-Spytag | spytag003 |
| RW90 | P. putida RW87 pBTL-2 | OmpA from E. coli was fused to spycatcher003 for |
| | ompA_{Ec}-spycatcher | internal display of cargo in the OMV |
| RW91 | P. putida RW87 pBTL-2 | INP from P. syringae was fused to spycatcher003 for |
| | inp_{Ps}-spycatcher | external display of cargo in the OMV |
| RW92 | P. putida RW87 PP_1122- | PP_1122 was truncated and fused to spycatcher003 for |
| | spycatcher | internal display of cargo in the OMV |
| RW93 | P. putida RW87 estP-spycatcher | EstP was truncated and fused to spycatcher003 for |
| | | external display of cargo in the OMV |

In an embodiment, disclosed herein are the use of different vesicle nucleating peptide (VNp) tags to enhance vesiculation and also target enzymes into OMVs (e.g. the fluorescent protein mNG) in Pseudomonas sp. In an embodiment disclosed herein is a system for export of recombinant proteins of interest in membrane-bound vesicles from Pseudomonas sp. In an embodiment the Pseudomonas sp. used in the system includes genetically modified P. putida, P. fluorescens, and P. stutzeri. In an embodiment, the system uses a peptide tag (VNp) that allows high-yield production of proteins of interest within vesicle packages that simplifies purification and enables long-term storage. In an embodiment, the system uses a peptide tag (VNp) that is linked to a protein of interest within vesicle packages and thus simplifies purification and enables long-term storage. This approach allows for the production of insoluble, toxic, and otherwise challenging proteins from Pseudomonas sp. In an embodiment, VNp tags can enhance the production of OMVs and can load the OMVs with enzymes or other proteins of interest. Using VNp tags allows for the modulation and enhancement of protein secretion through OMVs.

In an embodiment, OMV size measurements from TM27, TM26, and TM35 were measured through dynamic light scattering. The two largest peak populations may be depicted as the average diameters in nanometers of the OMVs in a first peak and a second peak wherein the population fractions may be depicted as peak area percentages.

Table 5 discloses the amino acid sequences of VNp tags disclosed herein.

TABLE 5

| Tag | Amino Acid sequence |
|---|---|
| VNp | (SEQ ID NO: 5) |
| | MDVFMKGLSKAKEGVVAAAE |
| | KTKQGVAEAAGKTKEGVL |
| VNp6 | (SEQ ID NO: 6) |
| | MDVFKKGFSIADEGVVGAVE |
| | KTDQGVTEAAEKTKEGVM |
| VNp15 | (SEQ ID NO: 7) |
| | MDVFKKGFSIADEGVVGAVE |

Table 6 discloses different strains of P. putida disclosed herein. Each strain is genetically modified to carry a different type of VNp tag linked to a fluorescent protein, a fluorescent protein, or no tag or protein at all.

TABLE 6

| Strain | Genotype | Details |
|---|---|---|
| TM35 | P. putida KT2440 carrying pBTL2 empty vector | Control used, same antibiotic |
| | | resistance |
| TM26 | P. putida KT2440 carrying pBTL2 with mNG- | Expression of fluorescent |
| | 6His | mNeongreen (mNG) with a 6his |
| | | tag |
| TM27 | P. putida KT2440 carrying pBTL2 with VNp- | Expression of mNG-his linked to |
| | mNG-6His | VNp |
| TM28 | P. putida KT2440 carrying pBTL2 with VNp6- | Expression of mNG-his linked to |
| | mNG-6His | VNp6 |
| TM29 | P. putida KT2440 carrying pBTL2 with VNp15- | Expression of mNG-his linked to |
| | mNG-6His | VNp15 |

### Materials and Methods

### Bacterial Strains and Media.

The strains used herein include P. putida KT2440 (ATCC 47054) and genetically engineered derivatives of this strain. Gene disruptions were verified by Sanger sequencing the associated molecular barcodes. All strains were stored in 25% glycerol at −80° C. Strains were revived by directly inoculating frozen stocks into Luria-Bertani (LB) medium (Lennox) at 30° C. Cells were cultivated in either LB medium or a modified M9 minimal media (6.78 g/L Na₂HPO₄ 3 g/L KH₂PO₄, 0.5 g/L NaCl, 1 g/L NH₄Cl, 2 mM MgSO₄, 100 μM CaCl₂), and 18 μM FeSO₄). Glucose was supplemented, as described for each experiment, into the M9 minimal media from a filtered 2 M solution in water to a final concentration of 20 mM or 50 mM. All media with p-coumarate and ferulate were titrated with 5 M NaOH to solubilize and neutralize to a final pH of 7.0. For aromatic compound tolerance experiments, 200 mM of p-coumarate or ferulate was made in 20 mM glucose M9 minimal media and diluted to the tested aromatic compound concentrations (200 mM, 125 mM, 75 mM, 25 mM, 0 mM). For shake flask experiments, a stock solution of 25 mM p-coumarate and ferulate was solubilized in M9 salts (6.78 g/L Na₂HPO₄ 3 g/L KH₂PO₄, 0.5 g/L NaCl, 1 g/L NH₄Cl) before mixing with the other components to make a final concentration of 20 mM glucose, 12.5 mM p-coumarate, and 12.5 mM ferulate in M9 minimal media. Pseudomonas quinolone signal (2-heptyl-3-hydroxy-4 (1H)-quinolone; PQS) was purchased from Sigma-Aldrich (Cat. #108985-27-9) and prepared in methanol at a stock concentration of 5 mM. For experimental conditions containing PQS, the stock was spiked into individual flasks to a final concentration of 50 μM and the methanol was evaporated in each flask under sterile conditions overnight before addition of experimental minimal media. All media filter sterilized (0.2 μm pore size) before use.

### Plasmid and Strain Construction.

Construction details including plasmids, oligonucleotides, and strains are detailed in Tables 1-6. In brief, gene knockouts using pK18sB plasmids and 1000 base pair homology regions were synthesized by Twist Biosciences. Competent P. putida cells were prepared before electroporation with the 500 ng of plasmid DNA. Cells were recovered for 1-2 hours in SOC media (0.2 g/L tryptone, 0.05 g/L yeast extract, 10 mM NaCl, 2.5 mM KCl, 10 mM MgCl₂, 10 mM MgSO₄, and 20 mM glucose) at 30° C. Markerless gene deletion was accomplished by the sacB/KanR counterselection. Correct transformants, either deletions or integrations, were screened with colony polymerase chain reaction (cPCR) and confirmed with Sanger sequencing at GENEWIZ (Azenta USA) or Oxford Nanopore sequencing at Plasmidsaurus. Correct colonies were stored as 20% (v/v) glycerol stocks at −80° C.

### Bacterial Growth.

Seed cultures from glycerol stocks were revived in LB medium until late exponential phase, pelleted at 5,000 g for 5 min, washed in M9 salts, pelleted again, and resuspended in the experimental M9 minimal media at an optical density, measured at 600 nm, (OD₆₀₀) of ˜0.1. Cells were grown in 100-well honeycomb plates (Growth Curves USA, part #9502550), culture tubes, or baffled flasks (either 150-mL or 250-mL) depending on the experiment. In all cases, cultures were grown at 30° C. with shaking to provide high aeration. For aromatic compound tolerance experiments, cells were grown in the honeycomb plates for 30 h in the BioscreenC Pro instrument (Growth Curves Ltd.) at maximum shaking speed with OD₆₀₀ measurements every 15 min. For consumption profiling, OMV extractions, and hydroxyacid production, cells were grown in shake flasks at one-fifth total flask volume. Aliquots of cell suspensions were collected for monitoring growth at OD₆₀₀ and quantifying extracellular metabolites. Samples for metabolite quantification were collected by centrifuging for 5 min at >10,000 g and filtering the supernatant through 0.22 μm nylon Costar Spin-X centrifuge tube filters (Corning). All extracellular metabolite samples were stored at −20° C. until analysis.

### OMV Extraction and Quantification.

Cells of P. putida and derivative strains were cultivated in M9 minimal media with glucose alone or glucose plus hydroxycinnamate compounds until mid-to-late exponential phase. An enrichment of OMVs was extracted from cell cultures as described previously. In brief, aliquots (25-30 mL) of cell culture were collected in sterile 50 mL centrifuge tubes and pelleted at 8,000 g for 20 min at 4° C. The supernatants were collected and transferred to new sterile 50 mL centrifuge tubes and spun again at 8,000 g for 20 min at 4° C. The resulting supernatant was filtered through a 0.2 μm filter unit (ThermoFisher Cat. #596-4520) to produce a cell-free clarified supernatant used for OMV extractions. Enrichment of OMVs from the clarified supernatant was conducted using the ExoBacteria OMV Isolation Kit (SBI Cat. #EXOBAC100A-1). The enriched OMV fraction, eluted in 1.5 mL, was analyzed directly or stored at −80° C. before quantification with nanoparticle tracking analysis (NTA). A maximum of one freeze-thaw cycle was conducted before analysis to minimize OMV lysis.

The enriched OMVs were 1:20 or 1:50 diluted to reach a particle concentration between 10∧7-10∧8 particles/mL using 0.22 μm-filtered PBS buffer. The samples were injected through a flow cell at a rate of 30 μl/min and analyzed on a NanoSight NS300 system (Malvern Panalytical, UK) equipped with a 638 nm laser with a 650 nm long-pass filter in the Analytical bioNanoTechnology Equipment Core Facility of the Simpson Querrey Institute for BioNanotechnology at Northwestern University. Each biological replicate was measured in three technical replicates. Data processing was performed on the Nanosight software (NTA 3.0).

### Membrane Permeability Assessment.

Cells were grown until mid-exponential in glucose only or glucose plus the hydroxycinnamate compounds. The OD600 of each culture was measured and recorded for normalization. To create a positive control for permeabilized cells, an aliquot of P. putida KT2440 at mid-exponential was incubated with 2% toluene for 30 min. All cell suspensions were pelleted at 5000 g for 5 min and resuspended in phosphate buffered saline (PBS) at a 2×concentration. A propidium iodide assay was conducted, as described previously by incubating 500 μL of the cell suspensions with 5 μL of propidium iodide solution (0.1 mg/mL in miliQ H₂O) for 10 min at room temperature. A 100 μL aliquot of each reacted cell suspension was transferred into a 96-well plate in technical duplicate. The fluorescence of DNA bound propidium iodide was measured on the Tecan microplate reader (Infinite® 200Pro, Tecan Group Ltd.) at an excitation of 535 nm and an emission of 617 nm with multiple reads per well (4×4).

### Quantification of Glucose and Aromatic Compounds.

Quantification of glucose and aromatic acids (p-coumaric acid, ferulic acid, 4-hydroxybenzoic acid, vanillic acid, 4-hydroxybenzaldehyde, vanillin, and protocatechuic acid) were analyzed. In brief, glucose was analyzed using an Agilent 1200 Series system performing high performance liquid chromatography with refractive index detection (HPLC-RID). Isocratic separation was conducted at a flow rate of 0.6 mL/min with a Bio-Rad Aminex HPX-87H Ion Exclusion Column (300×8.7 mm, 9 μm particle size) maintained at 55° C. For aromatic acids, reverse phase chromatographic separation was conducted on an Agilent 1290 series ultra-high performance liquid chromatography system combined with a diode array detector (UHPLC-DAD). A Phenomenex Kinetex reverse phase analytical column (2.1 mm×100 mm; 1.7 μm particle size) was utilized with a flow rate of 0.8 mL/min and the temperature maintained at 35° C. Linear calibration curves for each analyte of interest had an r² coefficient ≥0.995 and were used to quantify glucose and aromatic acids in the extracellular medium.

Table 7 lists nucleotide and amino acid sequences of some genes and proteins disclosed herein:

TABLE 7

| Strain of | | | |
|---|---|---|---|
| interest; | | | |
| Source; | | | |
| Accession | | | |
| no.; | | | |
| common | | | |
| name; | | | Amino acid |
| genotype | Notes | Nucleotide sequence | sequence |
| RW29; | ΔoprF | (SEQ ID NO: 8) | (SEQ ID NO: 9) |
| KT2440; | | atgAAACTGAAAAACACCTTGGGCT | MKLKNTLGLAIGSLV |
| PP_2089; | | TGGCCATTGGTTCGCTTGTAGCCGC | AATSIGAMAQGQGAV |
| oprF; | | CACTTCGATTGGCGCTATGGCACAA | ETEIFYKKEFFDSQR |
| KT2440 | | GGTCAAGGCGCCGTCGAGACTGAAA | DFKNDGNLFGGSIGY |
| ΔPP_2089 | | TCTTCTACAAGAAAGAATTCTTCGA | FLTDDVELRLGYDEV |
| | | CAGCCAGCGCGACTTCAAGAACGAC | HNARGEDGKNIKGSN |
| | | GGCAACCTGTTCGGCGGCTCGATCG | TALDAVYHENNPYDA |
| | | GTTACTTCCTGACCGACGACGTTGA | IRPYVSAGFSHQSLG |
| | | GCTGCGTCTGGGCTACGACGAAGTG | QTGRGGRDHSTFANV |
| | | CACAACGCTCGTGGCGAAGACGGCA | GAGAKWYITDMFYAR |
| | | AGAACATCAAGGGCTCGAACACTGC | AGVEAQYNIDQGDTE |
| | | CCTGGACGCCGTTTACCACTTCAAC | WAPSVGVGLNFGGSP |
| | | AACCCGTACGACGCTATCCGTCCAT | KQAEAAPAPVAEVCS |
| | | ACGTTTCCGCTGGTTTCTCGCACCA | DSDNDGVCDNVDKCP |
| | | GTCGCTGGGCCAGACCGGCCGTGGC | DTPANVTVDADGCPA |
| | | GGTCGTGACCACTCCACCTTCGCCA | VAEVVRVELDVKFDF |
| | | ACGTTGGCGCTGGCGCCAAGTGGTA | DKSVVKPNSYGDIKN |
| | | CATCACCGACATGTTCTATGCCCGT | LADFMKQYPQTTTVV |
| | | GCCGGCGTAGAAGCTCAGTACAACA | EGHTDSVGPDAYNQK |
| | | TCGACCAGGGCGACACCGAGTGGGC | LSERRANAVKQVLTQ |
| | | CCCGAGCGTTGGTGTTGGCCTGAAC | QYGVESSRVDSVGYG |
| | | TTCGGCGGTAGCCCGAAGCAAGCTG | ETRPVADNATEEGRA |
| | | AAGCTGCTCCTGCTCCAGTTGCTGA | VNRRVEAQVEAQAK* |
| | | AGTCTGCTCCGACTCCGACAACGAC | |
| | | GGCGTGTGCGACAACGTCGACAAGT | |
| | | GCCCAGACACCCCGGCCAACGTTAC | |
| | | CGTTGACGCCGACGGCTGCCCGGCT | |
| | | GTTGCCGAAGTCGTTCGCGTTGAGC | |
| | | TGGACGTCAAGTTCGACTTCGACAA | |
| | | GTCGGTCGTCAAGCCTAACAGCTAC | |
| | | GGCGACATCAAAAACCTGGCTGACT | |
| | | TCATGAAGCAGTACCCACAAACCAC | |
| | | CACCGTGGTTGAAGGTCACACTGAC | |
| | | TCCGTGGGTCCAGACGCTTACAACC | |
| | | AGAAGCTGTCCGAGCGTCGTGCCAA | |
| | | CGCTGTCAAGCAAGTGCTGACCCAG | |
| | | CAGTACGGCGTAGAATCCAGCCGTG | |
| | | TTGACTCGGTTGGCTACGGCGAAAC | |
| | | CCGTCCGGTTGCTGACAACGCCACC | |
| | | GAAGAAGGCCGTGCTGTCAACCGTC | |
| | | GCGTTGAAGCTCAGGTAGAAGCCCA | |
| | | GGCCAAGtaa | |
| RW30; | ΔoprI | (SEQ ID NO: 10) | (SEQ ID NO: 11) |
| KT2440; | | atgAACAACGTTCTGAAATTCTCTG | MNNVLKFSALALAAV |
| PP 2322; | | CTCTGGCTCTGGCCGCAGTTCTGGC | LATGCSSVSKETEAR |
| oprI; | | TACCGGTTGCAGCAGCGTATCCAAA | LTATEDAAARAQARA |
| KT2440 | | GAAACCGAAGCTCGTCTGACTGCGA | DEAYRKADDAMAAAQ |
| ΔPP 2322 | | CTGAAGACGCAGCAGCTCGCGCTCA | KAQQTADEANERALR |
| | | AGCTCGTGCTGACGAAGCCTACCGT | MLDKASRK* |
| | | AAGGCTGACGACGCAATGGCAGCCG | |
| | | CTCAGAAGGCTCAGCAGACCGCTGA | |
| | | CGAAGCCAACGAGCGCGCCCTGCGT | |
| | | ATGCTGGACAAAGCCAGCCGCAAGt | |
| | | aa | |
| N/A; N/A; | | (SEQ ID NO: 12) | (SEQ ID NO: 13) |
| N/A; xylE | | atgaacaaaggtgtaatgcgaccgg | MNKGVMRPGHVQLRV |
| | | gccatgtgcagctgcgtgtactgga | LDMSKALEHYVELLG |
| | | catgagcaaggccctggaacactac | LIEMDRDDQGRVYLK |
| | | gtcgagttgctgggcctgatcgaga | AWTEVDKFSLVLREA |
| | | tggaccgtgacgaccagggccgtgt | DEPGMDFMGFKVVDE |
| | | ctatctgaaggcttggaccgaagtg | DALRQLERDLMAYGC |
| | | gataagttttccctggtgctacgcg | AVEQLPAGELNSCGR |
| | | aggctgacgagccgggcatggattt | RVRFQAPSGHHFELY |
| | | tatgggtttcaaggttgtggatgag | ADKEYTGKWGLNDVN |
| | | gatgctctccggcaactggagcggg | PEAWPRDLKGMAAVR |
| | | atctgatggcatatggctgtgccgt | FDHALMYGDELPATY |
| | | tgagcagctacccgcaggtgaactg | DLFTKVLGFYLAEQV |
| | | aacagttgtggccggcgcgtgcgct | LDENGTRVAQFLSLS |
| | | tccaggccccctccgggcatcactt | TKAHDVAFIHHPEKG |
| | | cgagttgtatgcagacaaggaatat | RLHHVSFHLETWEDL |
| | | actggaaagtggggtttgaatgacg | LRAADLISMTDTSID |
| | | tcaatcccgaggcatggccgcgcga | IGPTRHGLTHGKTIY |
| | | tctgaaaggtatggcggctgtgcgt | FFDPSGNRNEVFCGG |
| | | ttcgaccacgccctcatgtatggcg | DYNYPDHKPVTWTTD |
| | | acgaattgccggcgacctatgacct | QLGKAIFYHDRILNE |
| | | gttcaccaaggtgctcggtttctat | RFMTVLT* |
| | | ctggccgaacaggtgctggacgaaa | |
| | | atggcacgcgcgtcgcccagtttct | |
| | | cagtctgtcgaccaaggcccacgac | |
| | | gtggccttcattcaccatccggaaa | |
| | | aaggccgcctccatcatgtgtcctt | |
| | | ccacctcgaaacctgggaagacttg | |
| | | cttcgcgccgccgacctgatctcca | |
| | | tgaccgacacatctatcgatatcgg | |
| | | cccaacccgccacggcctcactcac | |
| | | ggcaagaccatctacttcttcgacc | |
| | | cgtccggtaaccgcaacgaagtgtt | |
| | | ctgcgggggagattacaactacccg | |
| | | gaccacaaaccggtgacctggacca | |
| | | ccgaccagctgggcaaggcgatctt | |
| | | ttaccacgaccgcattctcaacgaa | |
| | | cgattcatgaccgtgctgacctga | |
| RW87; | Parent | (SEQ ID NO: 14) | (SEQ ID NO: 15) |
| xylE_ST; | strain | CGTGGCGTTCCTCATATTGTTATGG | RGVPHIVMVDAYKRY |
| P. putida | with | TGGACGCCTACAAACGCTATAAAGG | KGGGSMNKGVMRPGH |
| KT2440 | cargo | CGGTGGTTCGATGAACAAAGGTGTA | VQLRVLDMSKALEHY |
| ΔcatA2 | protein | ATGCGACCGGGCCATGTGCAGCTGC | VELLGLIEMDRDDQG |
| ΔcatRBCA:: | (XylE) | GTGTACTGGACATGAGCAAGGCCCT | RVYLKAWTEVDKFSL |
| Ptac: XylE- | fused | GGAACACTACGTCGAGTTGCTGGGC | VLREADEPGMDFMGF |
| Spytag | to | CTGATCGAGATGGACCGTGACGACC | KVVDEDALRQLERDL |
| | spytag003 | AGGGCCGTGTCTATCTGAAGGCTTG | MAYGCAVEQLPAGEL |
| | | GACCGAAGTGGATAAGTTTTCCCTG | NSCGRRVRFQAPSGH |
| | | GTGCTACGCGAGGCTGACGAGCCGG | HFELYADKEYTGKWG |
| | | GCATGGATTTTATGGGTTTCAAGGT | LNDVNPEAWPRDLKG |
| | | TGTGGATGAGGATGCTCTCCGGCAA | MAAVRFDHALMYGDE |
| | | CTGGAGCGGGATCTGATGGCATATG | LPATYDLFTKVLGFY |
| | | GCTGTGCCGTTGAGCAGCTACCCGC | LAEQVLDENGTRVAQ |
| | | AGGTGAACTGAACAGTTGTGGCCGG | FLSLSTKAHDVAFIH |
| | | CGCGTGCGCTTCCAGGCCCCCTCCG | HPEKGRLHHVSFHLE |
| | | GGCATCACTTCGAGTTGTATGCAGA | TWEDLLRAADLISMT |
| | | CAAGGAATATACTGGAAAGTGGGGT | DTSIDIGPTRHGLTH |
| | | TTGAATGACGTCAATCCCGAGGCAT | GKTIYFFDPSGNRNE |
| | | GGCCGCGCGATCTGAAAGGTATGGC | VFCGGDYNYPDHKPV |
| | | GGCTGTGCGTTTCGACCACGCCCTC | TWTTDQLGKAIFYHD |
| | | ATGTATGGCGACGAATTGCCGGCGA | RILNERFMTVLT* |
| | | CCTATGACCTGTTCACCAAGGTGCT | |
| | | CGGTTTCTATCTGGCCGAACAGGTG | |
| | | CTGGACGAAAATGGCACGCGCGTCG | |
| | | CCCAGTTTCTCAGTCTGTCGACCAA | |
| | | GGCCCACGACGTGGCCTTCATTCAC | |
| | | CATCCGGAAAAAGGCCGCCTCCATC | |
| | | ATGTGTCCTTCCACCTCGAAACCTG | |
| | | GGAAGACTTGCTTCGCGCCGCCGAC | |
| | | CTGATCTCCATGACCGACACATCTA | |
| | | TCGATATCGGCCCAACCCGCCACGG | |
| | | CCTCACTCACGGCAAGACCATCTAC | |
| | | TTCTTCGACCCGTCCGGTAACCGCA | |
| | | ACGAAGTGTTCTGCGGGGGAGATTA | |
| | | CAACTACCCGGACCACAAACCGGTG | |
| | | ACCTGGACCACCGACCAGCTGGGCA | |
| | | AGGCGATCTTTTACCACGACCGCAT | |
| | | TCTCAACGAACGATTCATGACCGTG | |
| | | CTGACCTGA | |
| RW02; | ΔompA | (SEQ ID NO: 16) | (SEQ ID NO: 17) |
| KT2440; | | atgAGCATAGTACGCACAGCGTTAC | MSIVRTALPLVLLTS |
| PP_1122; | | CCCTGGTACTGCTCACCAGTGTGTT | VLTGCAGLQKTDWPK |
| ompA; | | GACTGGTTGTGCAGGTTTGCAAAAA | CAAVGGVGGAALGAI |
| KT2440 | | ACCGACTGGCCGAAATGTGCCGCCG | ESSSWAGWGALLGGG |
| PP_1122:: | | TCGGGGGTGTAGGCGGCGCCGCCCT | LAAGYCWAHGDGDED |
| Tc1₁₂₈₅₀₆₈(Km) | | GGGCGCCATCGAAAGCTCCAGCTGG | GDGVPDSRDKCPGTP |
| | | GCTGGCTGGGGTGCGTTGCTGGGCG | RGVQVDANGCPPEPV |
| | | GTGGCCTGGCGGCGGGCTATTGCTG | AVVEEVVVQKEEVIV |
| | | GGCCCATGGCGATGGCGACGAGGAT | IRDVHFEFDSARLTA |
| | | GGTGACGGCGTGCCAGACAGCCGTG | SDKERLNTIATRLKQ |
| | | ACAAGTGCCCTGGCACCCCGCGTGG | EAPSARLSVSGHTDS |
| | | TGTGCAGGTCGATGCCAACGGATGC | VGSDSYNQKLSERRA |
| | | CCGCCTGAGCCGGTTGCGGTGGTCG | HSVTDYLVESGVPRS |
| | | AAGAAGTGGTGGTGCAGAAGGAAGA | SFVSVVGAGETQPVA |
| | | AGTCATTGTCATCCGCGATGTGCAC | DNATAEGRAMNRRTE |
| | | TTCGAGTTCGATTCTGCGCGCCTGA | IKIQR* |
| | | CGGCCAGTGACAAAGAGCGCCTCAA | |
| | | TACCATTGCCACGCGCCTGAAGCAG | |
| | | GAAGCGCCCTCTGCCCGCCTTAGCG | |
| | | TCAGCGGCCATACCGACAGCGTCGG | |
| | | TTCCGACAGCTACAACCAGAAACTG | |
| | | TCCGAGCGCCGTGCCCATTCGGTGA | |
| | | CCGATTACCTGGTCGAGAGCGGTGT | |
| | | ACCGCGCAGCAGCTTCGTTTCGGTG | |
| | | GTCGGCGCGGGTGAAACCCAGCCGG | |
| | | TGGCAGACAACGCCACGGCCGAAGG | |
| | | GCGTGCCATGAACCGTCGTACCGAG | |
| | | ATCAAGATCCAGCGGtaa | |
| RW92; | PP_1122 | (SEQ ID NO: 18) | (SEQ ID NO: 19) |
| KT2440; | was | gtgCCTCGAGCAGTGGCACGGGCGC | VPRAVARARLHAGQP |
| PP_1122; | truncated | GTCTTCATGCCGGCCAGCCGTTTGT | FVANDTGAFSMSVTS |
| ompA_trunc_ | and | GGCCAATGACACAGGAGCATTCAGC | KAALPLLVAASLLTG |
| SC; P. | fused | ATGAGTGTGACGTCGAAGGCGGCTT | CATHSDGSAPLNQRT |
| putida RW87 | to | TGCCGCTGTTGGTGGCTGCCAGCCT | WPICSLLGGLVGGGL |
| PP_1122- | spycat | GCTCACGGGCTGCGCTACGCACAGC | GAIESSSWAAGGGAL |
| spycatcher | cher003 | GATGGCAGCGCGCCCCTCAATCAAA | GAIAGGLICYAQDGD |
| | for | GGACCTGGCCCATCTGCAGCCTGCT | EDGDGIFDRRDHCPE |
| | internal | GGGCGGCTTGGTCGGTGGTGGCCTT | TPANTAVDHMGCPLK |
| | display | GGTGCCATCGAGAGTTCTTCCTGGG | QYPAAPPAGGGGSVT |
| | of | CCGCCGGTGGTGGCGCCTTGGGCGC | TLSGLSGEQGPSGDM |
| | cargo | CATTGCCGGCGGGCTGATTTGTTAC | TTEEDSATHIKFSKR |
| | in the | GCCCAGGACGGTGACGAAGATGGTG | DEDGRELAGATMELR |
| | OMV | ACGGCATTTTCGACCGGCGCGATCA | DSSGKTISTWISDGH |
| | | CTGCCCCGAGACCCCGGCCAACACG | VKDFYLYPGKYTFVE |
| | | GCGGTTGACCACATGGGCTGCCCAC | TAAPDGYEVATPIEF |
| | | TGAAACAGTACCCGGCCGCGCCACC | TVNEDGQVTVDGEAT |
| | | TGCCGGTGGTGGCGGGAGCgtaacc | EGDAHT |
| | | accttatcaggtttatcaggtgagc | |
| | | aaggtccgtccggtgatatgacaac | |
| | | tgaagaagatagtgctacccatatt | |
| | | aaattctcaaaacgtgatgaggacg | |
| | | gccgtgagttagctggtgcaactat | |
| | | ggagttgcgtgattcatctggtaaa | |
| | | actattagtacatggatttcagatg | |
| | | gacatgtgaaggatttctacctgta | |
| | | tccaggaaaatatacatttgtcgaa | |
| | | accgcagcaccagacggttatgagg | |
| | | tagcaactccaattgaatttacagt | |
| | | taatgaggacggtcaggttactgta | |
| | | gatggtgaagcaactgaaggtgacg | |
| | | ctcatact | |
| N/A; | | (SEQ ID NO: 20) | (SEQ ID NO: 21) |
| KT2440; | | atgCGAAAAGCCCCGTTATTGCGCT | MRKAPLLRFTLASLA |
| PP_0418; | | TTACCCTCGCTTCACTGGCCCTGGC | LACSQALAGPSPYST |
| EstP | | CTGCAGCCAGGCGTTGGCCGGCCCT | LIVFGDSLADAGQEP |
| | | TCGCCCTATTCAACCCTGATCGTGT | DLVGGTPGARFTNRD |
| | | TTGGCGACAGCCTCGCCGATGCCGG | ADGNFAPVSPMILGG |
| | | GCAGTTTCCCGATCTTGTTGGCGGT | RLGVAPGDLNPSTSV |
| | | ACCCCAGGCGCGCGTTTCACCAACC | GIQPDGNNWAVGGYT |
| | | GTGACGCCGACGGCAACTTCGCCCC | TQQILDSITTTSETV |
| | | GGTGTCGCCGATGATCCTCGGTGGC | IPPGNPNAGLVLRER |
| | | CGCCTGGGCGTCGCGCCAGGCGAAC | PGYLANGLRADPNAL |
| | | CTTAACCCGTCGACATCCGTAGGTA | YYLTGGGNDFLQGLV |
| | | TCCAGCCCGATGGTAATAACTGGGC | NSPADAVAAGARLAA |
| | | AGTCGGCGGGTACACCACCCAGCAG | SAQALQQGGARYIMV |
| | | ATCCTGGACTCGATCACGACAACGT | WLLPDLGQTPNFSGT |
| | | CCGAGACCGTCATCCCCCCAGGAAA | PQQNPLSLLSAAFNQ |
| | | CCCCAATGCCGGGTTGGTGCTGCGC | SLISQLGQIDAQLII |
| | | GAGCGCCCCGGCTACCTGGCCAACG | PLNIPLLLSEALASP |
| | | GCCTGCGCGCCGACCCCAATGCCTT | SQFGLASDQNLVGTC |
| | | GTACTACCTGACAGGCGGCGGCAAC | YSGDSCVENPVYGIN |
| | | GACTTCCTTCAGGGCCTGGTGAATA | GTTPDPTKLLENDSV |
| | | GCCCGGCCGACGCCGTAGCCGCCGG | HPTIAGQQLIADYAY |
| | | CGCCCGCCTGGCTGCCAGCGCCCAA | SILAAPWELTLLPEM |
| | | GCGCTTCAGCAAGGAGGCGCGCGCT | AHASLRAHQDELRNQ |
| | | ACATCATGGTCTGGCTGCTACCTGA | WQTPWQAVGQWQAFV |
| | | CCTCGGCCAAACGCCCAATTTCAGT | ASGAQDLDFDGQHSA |
| | | GGCACGCCACAGCAAAACCCACTGT | ASGDGRGYNLTVGGS |
| | | CACTGCTCTCCGCTGCGTTCAACCA | YRLNDAWRLGLAGGA |
| | | GTCACTGATCAGCCAGCTAGGGCAG | NRQKLEAGEQDSDYK |
| | | ATCGATGCCCAGATCATTCCACTGA | LNSYMASAFAQYRQD |
| | | ACATCCCTTTGCTGTTGAGCGAGGC | RWWADAALTAGHLDY |
| | | GCTGGCCAGCCCCAGTCAGTTCGGC | SDLKRTFALGVNDRS |
| | | CTGGCCAGCGACCAGAACCTGGTCG | EKGDTDGEAWAMSGR |
| | | GCACCTGCTATAGCGGCGATAGCTG | LGYNLAADTSNWQLA |
| | | CGTGGAAAACCCGGTGTACGGGATC | PFTSADYARVKVDGY |
| | | AACGGCACAACGCCAGACCCGACCA | DEKSGRSTALGFDDQ |
| | | AACTGCTGTTCAACGACTCGGTCCA | ERTSRRLGVGLLGSV |
| | | CCCGACCATCGCGGGTCAGCAGCTG | QVLPSTRLFAEVAQE |
| | | ATTGCCGATTACGCCTACTCGATCC | HEFEDDEQDVTMHLT |
| | | TCGCGGCCCCCTGGGAACTGACCCT | SLPANDFTLTGYTPH |
| | | GCTACCGGAAATGGCCCACGCCAGC | SDLTRASLGVSHELV |
| | | CTGCGGGCTCACCAGGATGAGTTGC | AGVHLRGNYNWRKSD |
| | | GTAATCAGTGGCAGACGCCTTGGCA | ELTQQGISVGVSVD |
| | | AGCAGTTGGCCAATGGCAAGCCTTT | F* |
| | | GTCGCCAGCGGCGCTCAGGACCTGG | |
| | | ACTTCGACGGCCAGCACAGCGCGGC | |
| | | CAGCGGTGACGGCCGCGGCTACAAC | |
| | | CTGACCGTGGGCGGCAGCTATCGCC | |
| | | TGAACGACGCCTGGCGCCTGGGCCT | |
| | | GGCCGGCGGTGCAAACCGGCAGAAG | |
| | | CTGGAAGCTGGTGAACAGGACTCGG | |
| | | ACTACAAGCTGAACAGTTATATGGC | |
| | | CAGTGCCTTTGCCCAATACCGCCAG | |
| | | GACCGCTGGTGGGCCGACGCGGCGC | |
| | | TGACCGCCGGGCACCTGGATTACAG | |
| | | CGACCTCAAGCGTACCTTCGCCCTG | |
| | | GGCGTGAATGACCGCAGTGAGAAGG | |
| | | GCGACACCGACGGCGAGGCCTGGGC | |
| | | AATGTCCGGGCGGCTGGGCTACAAC | |
| | | CTGGCGGCCGACACCAGCAACTGGC | |
| | | AGTTGGCACCTTTCATCAGTGCCGA | |
| | | CTATGCGCGGGTGAAGGTGGATGGC | |
| | | TACGACGAGAAGAGCGGACGTTCGA | |
| | | CGGCGCTTGGCTTCGATGACCAGGA | |
| | | GCGCACGTCACGCCGCCTGGGCGTG | |
| | | GGGCTGCTGGGCAGTGTGCAGGTAC | |
| | | TGCCAAGTACCCGGCTTTTCGCCGA | |
| | | GGTGGCGCAGGAGCATGAGTTCGAG | |
| | | GACGACGAGCAGGATGTGACGATGC | |
| | | ACCTGACCAGCTTGCCGGCGAATGA | |
| | | CTTCACCCTGACCGGGTATACGCCG | |
| | | CACAGCGACCTGACCCGGGCGAGCC | |
| | | TGGGTGTGAGCCATGAACTGGTGGC | |
| | | AGGGGTGCATTTGCGCGGGAACTAC | |
| | | AACTGGCGCAAGAGTGATGAGTTGA | |
| | | CGCAACAGGGTATTAGCGTGGGGGT | |
| | | TAGCGTGGACTTCtga | |
| RW93; | EstP | (SEQ ID NO: 22) | (SED ID NO: 23) |
| KT2440; | was | gtaaccaccttatcaggtttatcag | VTTLSGLSGEQGPSG |
| PP_0418; | truncated | gtgagcaaggtccgtccggtgatat | DMTTEEDSATHIKFS |
| estP_Trunc_ | and | gacaactgaagaagatagtgctacc | KRDEDGRELAGATME |
| SC; P. | fused | catattaaattctcaaaacgtgatg | LRDSSGKTISTWISD |
| putida RW87 | to | aggacggccgtgagttagctggtgc | GHVKDFYLYPGKYTF |
| estP | spycat | aactatggagttgcgtgattcatct | VETAAPDGYEVATPI |
| spycatcher | cher003 | ggtaaaactattagtacatggattt | EFTVNEDGQVTVDGE |
| | for | cagatggacatgtgaaggatttcta | ATEGDAHTGGGGSPT |
| | external | cctgtatccaggaaaatatacattt | IAGQQLIADYAYSIL |
| | display | gtcgaaaccgcagcaccagacggtt | AAPWELTLLPEMAHA |
| | of | atgaggtagcaactccaattgaatt | SLRAHQDELRNQWQT |
| | cargo | tacagttaatgaggacggtcagett | PWQAVGOWQAFVASG |
| | in the | actgtagatggtgaagcaactgaag | AQDLDFDGQHSAASG |
| | OMV | gtgacgctcatactGGTGGTGGGGG | DGRGYNLTVGGSYRL |
| | | CTCCCCGACCATCGCGGGTCAGCAG | NDAWRLGLAGGANRQ |
| | | CTGATTGCCGATTACGCCTACTCGA | KLEAGEQDSDYKLNS |
| | | TCCTCGCGGCCCCCTGGGAACTGAC | YMASAFAQYRQDRWW |
| | | CCTGCTACCGGAAATGGCCCACGCC | ADAALTAGHLDYSDL |
| | | AGCCTGCGGGCTCACCAGGATGAGT | KRTFALGVNDRSEKG |
| | | TGCGTAATCAGTGGCAGACGCCTTG | DTDGEAWAMSGRLGY |
| | | GCAAGCAGTTGGCCAATGGCAAGCC | NLAADTSNWOQLAPF |
| | | TTTGTCGCCAGCGGCGCTCAGGACC | ISADYARVKVDGYDE |
| | | TGGACTTCGACGGCCAGCACAGCGC | KSGRSTALGFDDQER |
| | | GGCCAGCGGTGACGGCCGCGGCTAC | TSRRLGVGLLGSVQV |
| | | AACCTGACCGTGGGCGGCAGCTATC | LPSTRLFAEVAQEHE |
| | | GCCTGAACGACGCCTGGCGCCTGGG | FEDDEQDVTMHLTSL |
| | | CCTGGCCGGCGGTGCAAACCGGCAG | PANDFTLTGYTPHSD |
| | | AAGCTGGAAGCTGGTGAACAGGACT | LTRASLGVSHELVAG |
| | | CGGACTACAAGCTGAACAGTTATAT | VHLRGNYNWRKSDEL |
| | | GGCCAGTGCCTTTGCCCAATACCGC | TQQGISVGVSVDF* |
| | | CAGGACCGCTGGTGGGCCGACGCGG | |
| | | CGCTGACCGCCGGGCACCTGGATTA | |
| | | CAGCGACCTCAAGCGTACCTTTCGC | |
| | | CCTGGGCGTGAATGACCGCAGTGAG | |
| | | AAGGGCGACACCGACGGCGAGGCCT | |
| | | GGGCAATGTCCGGGCGGCTGGGCTA | |
| | | CAACCTGGCGGCCGACACCAGCAAC | |
| | | TGGCAGTTGGCACCTTTCATCAGTG | |
| | | CCGACTATGCGCGGGTGAAGGTGGA | |
| | | TGGCTACGACGAGAAGAGCGGACGT | |
| | | TCGACGGCGCTTGGCTTCGATGACC | |
| | | AGGAGCGCACGTCACGCCGCCTGGG | |
| | | CGTGGGGCTGCTGGGCAGTGTGCAG | |
| | | GTACTGCCAAGTACCCGGCTTTTCG | |
| | | CCGAGGTGGCGCAGGAGCATGAGTT | |
| | | CGAGGACGACGAGCAGGATGTGACG | |
| | | ATGCACCTGACCAGCTTGCCGGCGA | |
| | | ATGACTTCACCCTGACCGGGTATAC | |
| | | GCCGCACAGCGACCTGACCCGGGCG | |
| | | AGCCTGGGTGTGAGCCATGAACTGG | |
| | | TGGCAGGGGTGCATTTGCGCGGGAA | |
| | | CTACAACTGGCGCAAGAGTGATGAG | |
| | | TTGACGCAACAGGGTATTAGCGTGG | |
| | | GGGTTAGCGTGGACTTCtga | |
| N/A; | full | (SEQ ID NO: 24) | (SEQ ID NO: 25) |
| ompAEc | length | atgAAAAAGACAGCTATCGCGATTG | MKKTAIAIAVALAGF |
| | ompA | CAGTGGCACTGGCTGGTTTCGCTAC | ATVAQAAPKDNTWYT |
| | from | CGTAGCGCAGGCCGCTCCGAAAGAT | GAKLGWSQYHDTGFI |
| | E. coli | AACACCTGGTACACTGGTGCTAAAC | NNNGPTHENQLGAGA |
| | | TGGGCTGGTCCCAGTACCATGACAC | FGGYQVNPYVGFEMG |
| | | TGGTTTCATCAACAACAATGGCCCG | YDWLGRMPYKGSVEN |
| | | ACCCATGAAAACCAACTGGGCGCTG | GAYKAQGVQLTAKLG |
| | | GTGCTTTTGGTGGTTACCAGGTTAA | YPITDDLDIYTRLGG |
| | | CCCGTATGTTGGCTTTGAAATGGGT | MVWRADTKSNVYGKN |
| | | TACGACTGGTTAGGTCGTATGCCGT | HDTGVSPVFAGGVEY |
| | | ACAAAGGCAGCGTTGAAAACGGTGC | AITPEIATRLEYQWT |
| | | ATACAAAGCTCAGGGCGTTCAACTG | NNIGDAHTIGTRPDN |
| | | ACCGCTAAACTGGGTTACCCAATCA | GMLSLGVSYRFGQGE |
| | | CTGACGACCTGGACATCTACACTCG | AAPVVAPAPAPAPEV |
| | | TCTGGGTGGTATGGTATGGCGTGCA | QTKHFTLKSDVLFNF |
| | | GACACTAAATCCAACGTTTATGGTA | NKATLKPEGQAALDQ |
| | | AAAACCACGACACCGGCGTTTCTCC | LYSQLSNLDPKDGSV |
| | | GGTCTTCGCTGGCGGTGTTGAGTAC | VVLGYTDRIGSDAYN |
| | | GCGATCACTCCTGAAATCGCTACCC | QGLSERRAQSVVDYL |
| | | GTCTGGAATACCAGTGGACCAACAA | ISKGIPADKISARGM |
| | | CATCGGTGACGCACACACCATCGGC | GESNPVTGNTCDNVK |
| | | ACTCGTCCGGACAACGGCATGCTGA | QRAALIDCLAPDRRV |
| | | GCCTGGGTGTTTCCTACCGTTTCGG | EIEVKGIKDVVTQPQ |
| | | TCAGGGCGAAGCAGCTCCAGTAGTT | A* |
| | | GCTCCGGCTCCAGCTCCGGCACCGG | |
| | | AAGTACAGACCAAGCACTTCACTCT | |
| | | GAAGTCTGACGTTCTGTTCAACTTC | |
| | | AACAAAGCAACCCTGAAACCGGAAG | |
| | | GTCAGGCTGCTCTGGATCAGCTGTA | |
| | | CAGCCAGCTGAGCAACCTGGATCCG | |
| | | AAAGACGGTTCCGTAGTTGTTCTGG | |
| | | GTTACACCGACCGCATCGGTTCTGA | |
| | | CGCTTACAACCAGGGTCTGTCCGAG | |
| | | CGCCGTGCTCAGTCTGTTGTTGATT | |
| | | ACCTGATCTCCAAAGGTATCCCGGC | |
| | | AGACAAGATCTCCGCACGTGGTATG | |
| | | GGCGAATCCAACCCGGTTACTGGCA | |
| | | ACACCTGTGACAACGTGAAACAGCG | |
| | | TGCTGCACTGATCGACTGCCTGGCT | |
| | | CCGGATCGTCGCGTAGAGATCGAAG | |
| | | TTAAAGGTATCAAAGACGTTGTAAC | |
| | | TCAGCCGCAGGCTtaa | |
| RW90; | OmpA | (SEQ ID NO: 26) | (SEQ ID NO: 27) |
| E. Coli; | from | atgAAAAAGACAGCTATCGCGATTG | MKKTAIAIAVALAGF |
| ompAEc; | E. coli | CAGTGGCACTGGCTGGTTTCGCTAC | ATVAQAAPKDNTWYT |
| P. putida | was | CGTAGCGCAGGCCGCTCCGAAAGAT | GAKLGWSQYHDTGFI |
| RW87 | fused | AACACCTGGTACACTGGTGCTAAAC | NNNGPTHENQLGAGA |
| pBTL-2 | to | TGGGCTGGTCCCAGTACCATGACAC | FGGYQVNPYVGFEMG |
| ompAEc- | spycat | TGGTTTCATCAACAACAATGGCCCG | YDWLGRMPYKGSVEN |
| spycatcher | cher003 | ACCCATGAAAACCAACTGGGCGCTG | GAYKAQGVQLTAKLG |
| | for | GTGCTTTTGGTGGTTACCAGGTTAA | YPITDDLDIYTRLGG |
| | internal | CCCGTATGTTGGCTTTGAAATGGGT | MVWRADTKSNVYGKN |
| | display | TACGACTGGTTAGGTCGTATGCCGT | HDTGVSPVFAGGVEY |
| | of | ACAAAGGCAGCGTTGAAAACGGTGC | AITPEIATRLEYQWT |
| | cargo | ATACAAAGCTCAGGGCGTTCAACTG | NNIGDAHTIGTRPDN |
| | in the | ACCGCTAAACTGGGTTACCCAATCA | GMLSLGVSYRFGGGG |
| | OMV | CTGACGACCTGGACATCTACACTCG | GSVTTLSGLSGEQGP |
| | | TCTGGGTGGTATGGTATGGCGTGCA | SGDMTTEEDSATHIK |
| | | GACACTAAATCCAACGTTTATGGTA | FSKRDEDGRELAGAT |
| | | AAAACCACGACACCGGCGTTTCTCC | MELRDSSGKTISTWI |
| | | GGTCTTCGCTGGCGGTGTTGAGTAC | SDGHVKDFYLYPGKY |
| | | GCGATCACTCCTGAAATCGCTACCC | TFVETAAPDGYEVAT |
| | | GTCTGGAATACCAGTGGACCAACAA | PIEFTVNEDGQVTVD |
| | | CATCGGTGACGCACACACCATCGGC | GEATEGDAHT |
| | | ACTCGTCCGGACAACGGCATGCTGA | |
| | | GCCTGGGTGTTTCCTACCGTTTCGG | |
| | | TGGCGGTGGGGGTTCGgtaaccacc | |
| | | ttatcaggtttatcaggtgagcaag | |
| | | gtccgtccggtgatatgacaactga | |
| | | agaagatagtgctacccatattaaa | |
| | | ttctcaaaacgtgatgaggacggcc | |
| | | gtgagttagctggtgcaactatgga | |
| | | gttgcgtgattcatctggtaaaact | |
| | | attagtacatggatttcagatggac | |
| | | atgtgaaggatttctacctgtatcc | |
| | | aggaaaatatacatttgtcgaaacc | |
| | | gcagcaccagacggttatgaggtag | |
| | | caactccaattgaatttacagttaa | |
| | | tgaggacggtcaggttactgtagat | |
| | | ggtgaagcaactgaaggtgacgctc | |
| | | atact | |
| N/A; | | (SEQ ID NO: 28) | (SEQ ID NO: 29) |
| P. syringae; | | atgactctcgacaaggcgttggtgc | MTLDKALVLRTCANN |
| INP | | tgcgtacctgtgcaaataacatggc | MADHCGLIWPASGTV |
| (P. syringae) | | cgatcactgcggccttatatggccc | ESRYWQSTRRHENGL |
| | | gcgtccggcacggtggaatccagat | VGLLWGAGTSAFLSV |
| | | actggcagtcaaccaggcggcatga | HADARWIVCEVAVAD |
| | | gaatggtctggtcggtttactgtgg | IISLEEPGMVKFPRA |
| | | ggcgctggaaccagcgcttttctaa | EVVHVGDRISASHFI |
| | | gcgtgcatgccgatgctcgatggat | SARQADPASTSTSTS |
| | | tgtctgtgaagttgccgttgcagac | TSTLTPMPTAIPTPM |
| | | atcatcagtctggaagagccgggaa | PAVASVTLPVAEQAR |
| | | tggtcaagtttccgcgggccgaggt | HEVFDVASVSAAAAP |
| | | ggttcatgtcggcgacaggatcagc | VNTLPVTTPQNLQTA |
| | | gcgtcacacttcatttcggcacgtc | TYGSTLSGDNHSRLI |
| | | aggccgaccctGCGAGCACCAGCAC | AGYGSNETAGNHSDL |
| | | GTCCACGAGCACGAGCACGttaacg | IGGHDCTLMAGDQSR |
| | | ccaatgcctacggccatacccacgc | LTAGKNSVLTAGARS |
| | | ccatgcctgcggtagcaagtgtcac | KLIGSEGSTLSAGED |
| | | gttaccggtggccgaacaggcccgt | STLIFRLWDGKRYRQ |
| | | catgaagtgttcgatgtcgcgtcgg | LVARTGENGVEADIP |
| | | tcagcgcggctgccgccccagtaaa | YYVNEDDDIVDKPDE |
| | | caccctgccggtgacgacgccgcag | DDDWIEVK |
| | | aatttgcagaccgccacttacggca | |
| | | gcacgttgagtggcgacaatcacag | |
| | | tcgtctgattgccggttatggcagt | |
| | | aacgagaccgctggcaaccacagtg | |
| | | atctaattggcgggcatgactgcac | |
| | | cctgatggcgggagaccaaagcaga | |
| | | ttgaccgctggtaagaacagtgtct | |
| | | tgacggcaggcgctcgtagcaaact | |
| | | tattggcagtgaaggctcgacgctc | |
| | | tcggctggagaagactccacactaa | |
| | | ttttcagactctgggacgggaagag | |
| | | gtacaggcaactggtcgccagaacg | |
| | | ggtgagaacggtgttgaggccgaca | |
| | | taccgtattacgtgaacgaagatga | |
| | | cgatattgtcgataaacccgacgag | |
| | | gacgatgactggatagaggtaaag | |
| RW91; | INP | (SEQ ID NO: 30) | (SEQ ID NO: 31) |
| P. syringae; | from | atgactctcgacaaggcgttggtgc | MTLDKALVLRTCANN |
| INP_SC; | P. | tgcgtacctgtgcaaataacatggc | MADHCGLIWPASGTV |
| P. putida | syringae | cgatcactgcggccttatatggccc | ESRYWQSTRRHENGL |
| RW87 | was | gcgtccggcacggtggaatccagat | VGLLWGAGTSAFLSV |
| pBTL-2 | fused | actggcagtcaaccaggcggcatga | HADARWIVCEVAVAD |
| inpPs- | to | gaatggtctggtcggtttactgtgg | IISLEEPGMVKFPRA |
| spycatcher | spycat | ggcgctggaaccagcgcttttctaa | EVVHVGDRISASHFI |
| | cher003 | gcgtgcatgccgatgctcgatggat | SARQADPASTSTSTS |
| | for | tgtctgtgaagttgccgttgcagac | TSTLTPMPTAIPTPM |
| | external | atcatcagtctggaagagccgggaa | PAVASVTLPVAEQAR |
| | display | tggtcaagtttccgcgggccgaggt | HEVFDVASVSAAAAP |
| | of | ggttcatgtcggcgacaggatcagc | VNTLPVTTPQNLQTA |
| | cargo | gcgtcacacttcatttcggcacgtc | TYGSTLSGDNHSRLI |
| | in the | aggccgaccctGCGAGCACCAGCAC | AGYGSNETAGNHSDL |
| | OMV | GTCCACGAGCACGAGCACGttaacg | IGGHDCTLMAGDQSR |
| | | ccaatgcctacggccatacccacgc | LTAGKNSVLTAGARS |
| | | ccatgcctgcggtagcaagtgtcac | KLIGSEGSTLSAGED |
| | | gttaccggtggccgaacaggcccgt | STLIFRLWDGKRYRQ |
| | | catgaagtgttcgatgtcgcgtcgg | LVARTGENGVEADIP |
| | | tcagcgcggctgccgccccagtaaa | YYVNEDDDIVDKPDE |
| | | caccctgccggtgacgacgccgcag | DDDWIEVKGGGGSVT |
| | | aatttgcagaccgccacttacggca | TLSGLSGEQGPSGDM |
| | | gcacgttgagtggcgacaatcacag | TTEEDSATHIKFSKR |
| | | tcgtctgattgccggttatggcagt | DEDGRELAGATMELR |
| | | aacgagaccgctggcaaccacagtg | DSSGKTISTWISDGH |
| | | atctaattggcgggcatgactgcac | VKDFYLYPGKYTFVE |
| | | cctgatggcgggagaccaaagcaga | TAAPDGYEVATPIEF |
| | | ttgaccgctggtaagaacagtgtct | TVNEDGQVTVDGEAT |
| | | tgacggcaggcgctcgtagcaaact | EGDAHT |
| | | tattggcagtgaaggctcgacgctc | |
| | | tcggctggagaagactccacactaa | |
| | | ttttcagactctgggacgggaagag | |
| | | gtacaggcaactggtcgccagaacg | |
| | | ggtgagaacggtgttgaggccgaca | |
| | | taccgtattacgtgaacgaagatga | |
| | | cgatattgtcgataaacccgacgag | |
| | | gacgatgactggatagaggtaaagG | |
| | | GCGGTGGGGGTTCGgtaaccacctt | |
| | | atcaggtttatcaggtgagcaaggt | |
| | | ccgtccggtgatatgacaactgaag | |
| | | aagatagtgctacccatattaaatt | |
| | | ctcaaaacgtgatgaggacggccgt | |
| | | gagttagctggtgcaactatggagt | |
| | | tgcgtgattcatctggtaaaactat | |
| | | tagtacatggatttcagatggacat | |
| | | gtgaaggatttctacctgtatccag | |
| | | gaaaatatacatttgtcgaaaccgc | |
| | | agcaccagacggttatgaggtagca | |
| | | actccaattgaatttacagttaatg | |
| | | aggacggtcaggttactgtagatgg | |
| | | tgaagcaactgaaggtgacgctcat | |
| | | act | |
| RW03; | Δomp | (SEQ ID NO: 32) | (SEQ ID NO: 33) |
| KT2440; | A-like | ATGCGCAAACACGTAATGATTCCCG | MRKHVMIPALLALSV |
| PP_1502; | protein | CCCTGCTGGCCCTGAGCGTCGGTCT | GLAACSHDPNANLES |
| KT2440 | | TGCTGCCTGCTCGCATGATCCGAAT | ARTNFSSLQSDPQAS |
| PP_1502:: | | GCCAACCTGGAATCGGCCCGCACCA | KVAALETKDAQDWLN |
| Tc1₁₇₀₇₅₄₈(Km) | | ACTTCTCCTCACTGCAGAGCGACCC | KADKAYMDREDEKKV |
| | | GCAAGCGAGCAAAGTCGCGGCACTG | DQLAYLTNQRVEVAK |
| | | GAGACCAAGGACGCCCAGGACTGGC | QTIALRTAEAELKNA |
| | | TGAACAAGGCCGACAAGGCGTACAT | SAQRAQAKLDARDAQ |
| | | GGACCGTGAAGACGAGAAGAAAGTC | IAKLQDSLNAKQTDR |
| | | GACCAACTGGCCTACCTGACCAACC | GTLVTFGDVLFDFNK |
| | | AGCGCGTCGAAGTGGCCAAGCAGAC | AELKSNAYPNITKLA |
| | | CATTGCCCTGCGTACTGCCGAAGCT | QFLQENPERKVIVEG |
| | | GAACTGAAAAACGCCTCGGCCCAGC | YTDSVGSANYNQTLS |
| | | GCGCCCAGGCCAAGCTGGATGCCCG | ERRANSVRMALVRAG |
| | | CGACGCGCAGATCGCCAAGCTGCAG | VDPARIVSQGYGKEY |
| | | GACAGCCTCAACGCCAAGCAGACCG | PVADNSSNSGRAQNR |
| | | ACCGCGGAACGCTGGTGACCTTCGG | RVEVTISNDNQPVAP |
| | | CGACGTGCTGTTCGACTTCAACAAG | RSVSQVQR* |
| | | GCCGAACTTAAGAGCAACGCCTACC | |
| | | CGAACATCACCAAGCTGGCCCAGTT | |
| | | CCTTCAGGAAAACCCGGAACGCAAG | |
| | | GTGATCGTCGAGGGCTACACCGACA | |
| | | GCGTCGGCTCGGCCAACTACAACCA | |
| | | GACCCTGTCCGAGCGCCGTGCCAAC | |
| | | AGCGTGCGCATGGCACTGGTGCGTG | |
| | | CCGGGGTAGATCCGGCGCGTATCGT | |
| | | TTCCCAGGGCTATGGCAAGGAGTAC | |
| | | CCGGTAGCGGACAACTCGAGCAACT | |
| | | CGGGACGTGCGCAGAACCGTCGGGT | |
| | | GGAGGTGACCATCTCCAACGACAAC | |
| | | CAGCCGGTGGCACCACGCTCGGTGA | |
| | | GCCAGGTTCAGCGCTAA | |
| RW04; | Δomp | (SEQ ID NO: 34) | (SEQ ID NO: 35) |
| KT2440; | A-like | ATGATCCGTCGTACGCCTTTGGCTG | MIRRTPLAALALLAL |
| PP 4198; | protein | CACTGGCATTGCTGGCGCTGACGGC | TAGLQGCASQRSSAA |
| KT2440 | | AGGTTTGCAAGGTTGCGCCAGTCAG | LDEATVAFQGVKDDS |
| PP_4198:: | | CGCAGCAGTGCCGCGCTGGATGAGG | DVLRSAPRDVIRAGE |
| Tc1₄₇₄₄₂₄₄(Km) | | CCACCGTTGCCTTCCAGGGGGTCAA | SLARAERLSSYIGTG |
| | | AGATGATTCCGATGTGCTGCGCAGC | SDVRHYAYLSQRYSE |
| | | GCGCCGCGTGACGTGATTCGGGCGG | IAREHAKLALNQERQ |
| | | GTGAGTCGCTGGCTCGCGCCGAGCG | AKLDLERQRLQLALR |
| | | CCTGTCCAGTTACATCGGCACCGGT | EAKLASVQQQGKWVE |
| | | TCCGATGTGCGGCATTATGCTTACC | SQIAALASEQADRGL |
| | | TCAGCCAGCGCTACAGCGAGATTGC | VMTLGDVLFDTGSAD |
| | | CCGCGAGCATGCCAAGCTGGCGCTG | LKNSASRTVLKLVQF |
| | | AACCAGGAGCGCCAGGCCAAGCTCG | LQLNPRRVVRIEGYT |
| | | ACCTGGAGCGCCAGCGCCTGCAGCT | DSTGAGEENLKLSRD |
| | | GGCCTTGCGTGAGGCCAAACTGGCC | RAQSVADMLVDLGID |
| | | AGCGTGCAGCAGCAGGGCAAGTGGG | EKRLQVEGYGDQYPI |
| | | TCGAGTCGCAGATTGCCGCGTTGGC | EANASERGRAQNRRV |
| | | TTCGGAGCAGGCCGACCGTGGCTTG | EIVFSDDKGRLAPA |
| | | GTGATGACCTTGGGCGATGTGCTGT | R* |
| | | TCGATACCGGCAGTGCCGACCTGAA | |
| | | GAACTCGGCCAGCCGGACTGTGCTC | |
| | | AAGCTGGTGCAGTTCCTGCAGCTCA | |
| | | ACCCGCGCCGGGTAGTGCGTATTGA | |
| | | GGGCTATACCGACAGTACTGGCGCG | |
| | | GGCGAGGAGAATCTCAAGCTGTCGC | |
| | | GCGACCGGGCGCAGTCCGTGGCTGA | |
| | | CATGCTTGTGGACCTGGGCATCGAC | |
| | | GAAAAGCGCCTGCAGGTTGAAGGCT | |
| | | ATGGCGACCAGTACCCGATCGAGGC | |
| | | CAATGCTTCGGAGCGGGGCAGGGCG | |
| | | CAGAACCGTCGGGTGGAGATCGTAT | |
| | | TCTCCGATGACAAGGGGCGGCTCGC | |
| | | ACCGGCGCGCTGA | |
| RW27; | Δomp | (SEQ ID NO: 36) | (SEQ ID NO: 37) |
| KT2440; | A-like | atgAAATCAGGAACAGGAGTTGAAC | MKSGTGVEPVMHALR |
| PP_4669; | protein | CCGTGATGCACGCTTTACGTTTTCC | FPLWALLFAMLALTG |
| KT2440 | | GCTTTGGGCCTTGTTGTTCGCCATG | CQSAPQKGLTPEQIA |
| ΔPP_4669 | | CTGGCGCTGACGGGGTGCCAGAGCG | VLKREGFTPTDEGWA |
| | | CCCCCCAAAAGGGCCTTACCCCAGA | YDLSGKVLFGSDLDS |
| | | ACAGATTGCCGTGCTCAAGCGCGAA | LNGQSQAIVERIGKA |
| | | GGTTTCACCCCGACCGATGAAGGTT | LLGVGIQGVRVDGHA |
| | | GGGCCTACGACTTGTCTGGCAAAGT | DSSGKAAYNQQLSER |
| | | GCTGTTCGGCAGCGATCTGGACAGC | RAQSVTKALVGIGMQ |
| | | CTCAACGGCCAGAGCCAGGCGATTG | AQNIQSRGLGSSQPV |
| | | TCGAGCGCATCGGCAAGGCGCTGCT | ADNRTSAGRTENRRV |
| | | CGGCGTGGGTATCCAGGGCGTGCGG | SIVVASY* |
| | | GTGGACGGGCATGCCGACTCGTCGG | |
| | | GCAAGGCGGCGTATAACCAGCAGCT | |
| | | GTCCGAGCGCCGCGCGCAAAGCGTG | |
| | | ACCAAGGCGCTGGTGGGGATTGGCA | |
| | | TGCAGGCACAGAACATTCAGAGCCG | |
| | | TGGCCTGGGCAGCAGCCAGCCGGTG | |
| | | GCGGACAACCGCACCAGCGCCGGGC | |
| | | GTACCGAGAACCGTCGGGTGTCCAT | |
| | | CGTGGTAGCGTCCTACtga | |
| TM26, | | (SEQ ID NO: 38) | (SEQ ID NO: 39) |
| TM27, | | gtgagcaagggcgaggaggataaca | VSKGEEDNMASLPAT |
| TM28, | | tggcctctctcccagcgacacatga | HELHIFGSINGVDFD |
| TM29; | | gttacacatctttggctccatcaac | MVGQGTGNPNDGYEE |
| Branchiostoma | | ggtgtggactttgacatggtgggtc | LNLKSTKGDLQFSPW |
| lanceolatum; | | agggcaccggcaatccaaatgatgg | ILVPHIGYGFHQYLP |
| mNeongreen | | ttatgaggagttaaacctgaagtcc | YPDGMSPFQAAMVDG |
| (mNG) | | accaagggtgacctccagttctccc | SGYQVHRTMQFEDGA |
| | | cctggattctggtccctcatatcgg | SLTVNYRYTYEGSHI |
| | | gtatggcttccatcagtacctgccc | KGEAQVKGTGFPADG |
| | | taccctgacgggatgtcgcctttcc | PVMTNSLTAADWCRS |
| | | aggccgcgatggtagatggctccgg | KKTYPNDKTIISTFK |
| | | ataccaagtccatcgcacaatgcag | WSYTTGNGKRYRSTA |
| | | tttgaagatggtgcctcccttactg | RTTYTFAKPMAANYL |
| | | ttaactaccgctacacctacgaggg | KNQPMYVFRKTELKH |
| | | aagccacatcaaaggagaggcccag | SKTELNFKEWQKAFT |
| | | gtgaaggggactggtttccctgctg | DVMGMDELYK |
| | | acggtcctgtgatgaccaactcgct | |
| | | gaccgctgcggactggtgcaggtcg | |
| | | aagaagacttaccccaacgacaaaa | |
| | | ccatcatcagtacctttaagtggag | |
| | | ttacaccactggaaatggcaagcgc | |
| | | taccggagcactgcgcggaccacct | |
| | | acacctttgccaagccaatggcggc | |
| | | taactatctgaagaaccagccgatg | |
| | | tacgtgttccgtaagacggagctca | |
| | | agcactccaagaccgagctcaactt | |
| | | caaggagtggcaaaaggcctttacc | |
| | | gatgtgatgggcatggacgagctgt | |
| | | acaag | |

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

What is claimed is:

1. A genetically modified Pseudomonas sp. comprising at least one deletion of an endogenous gene, wherein:
the one or more deletion results in an increase in the production of outer membrane vesicles (OMVs) relative to the wild-type Pseudomonas sp.

2. The genetically modified Pseudomonas sp. of claim 1, wherein the endogenous gene is selected from the group consisting of oprF, and oprI.

3. The genetically modified Pseudomonas sp. of claim 1, wherein the Pseudomonas sp. is selected from the group consisting of P. putida, P. fluorescens, and P. stutzeri.

4. The genetically modified Pseudomonas sp. of claim 3, wherein the P. putida is P. putida KT2440.

5. A genetically modified Pseudomonas sp. comprising at least one deletion of an endogenous gene, wherein:
the one or more deletion results in an increase in the production of outer membrane vesicles (OMVs) relative to the wild-type Pseudomonas sp.; and
wherein the genetically modified Pseudomonas sp. further comprises at least one exogenous gene encoding an enzyme; and
wherein the expressed enzyme encoded by the at least one exogenous gene encoding an enzyme is connected to an outer membrane protein that is incorporated into the membrane of an outer membrane vesicle; and
wherein the enzyme is connected to the outer membrane protein through a linker.

6. The genetically modified Pseudomonas sp. of claim 5, wherein the expressed enzyme encoded by the at least one exogenous gene is tagged with a vesicle nucleating peptide.

7. The genetically modified Pseudomonas sp. of claim 5, wherein the expressed enzyme encoded by the at least one exogenous gene is tagged with a vesicle nucleating peptide having a sequence selected from the group consisting of SEQ ID NO: 5, SEQ ID NO: 6 and SEQ ID NO: 7.

8. A system for the production and isolation of a compound of interest comprising:
a genetically modified Pseudomonas sp. comprising at least one deletion of an endogenous gene, wherein:
the one or more deletion results in an increase in the production of outer membrane vesicles (OMVs) relative to the wild-type Pseudomonas sp.; and
wherein the genetically modified Pseudomonas sp. further comprises at least one exogenous gene encoding an enzyme; and
wherein the expressed enzyme encoded by the at least one exogenous gene encoding an enzyme is connected to an outer membrane protein that is incorporated into the membrane of an outer membrane vesicle; and
wherein the expressed enzyme is connected to the outer membrane protein through a linker; and
wherein the expressed enzyme encoded by the at least one exogenous gene is contacted with a substrate; and
wherein a product of a reaction catalyzed by the expressed enzyme encoded by the at least one exogenous gene is isolated; and
wherein the product of the reaction catalyzed by the expressed enzyme is the compound of interest.

9. The system of claim 8 wherein the expressed enzyme encoded by the at least one exogenous gene is XylE; and the substrate is catechol and the product is 2-hydroxymuconic semialdehyde.

10. The system of claim 8 wherein the expressed enzyme encoded by the at least one exogenous gene is isolated.

11. The system of claim 8 wherein the outer membrane protein is encoded by a gene that is endogenous to the genetically modified Pseudomonas sp.

12. The system of claim 11 wherein the outer membrane protein is selected from the group consisting of OmpA (PP_1122) and EstP.

13. The system of claim 12 wherein the outer membrane protein is OmpA (PP_1122) and wherein the expressed enzyme encoded by the at least one exogenous gene is on the inside of the outer membrane vesicle.

14. The system of claim 12 wherein the outer membrane protein is EstP and wherein the expressed enzyme encoded by the at least one exogenous gene is on the outside of the outer membrane vesicle.

15. The system of claim 8 wherein the outer membrane protein is encoded by a gene that is exogenous to the genetically modified Pseudomonas sp.

16. The system of claim 15 wherein the outer membrane protein is selected from the group consisting of OmpA from Escherichia coli or INP from Pseudomonas syringae.

17. The system of claim 16 wherein the outer membrane protein is OmpA from Escherichia coli and wherein the expressed enzyme encoded by the at least one exogenous gene is on the inside of the outer membrane vesicle.

18. The system of claim 16 wherein the outer membrane protein is INP from Pseudomonas syringae and wherein the expressed enzyme encoded by the at least one exogenous gene is on the outside of the outer membrane vesicle.

19. The system of claim 8 wherein the expressed enzyme encoded by the at least one exogenous gene is tagged with a vesicle nucleating peptide.

20. The system of claim 8 wherein the expressed enzyme encoded by the at least one exogenous gene is tagged with a vesicle nucleating peptide having a sequence selected from the group consisting of SEQ ID NO: 5, SEQ ID NO: 6 and SEQ ID NO: 7.


SEQUENCE LISTING (39 sequences)

SEQ ID NO: 1 - DNA, 20 bp, synthetic construct
  source 1..20: mol_type = other DNA
atcggcctggaatattcggc

SEQ ID NO: 2 - DNA, 19 bp, synthetic construct
  source 1..19: mol_type = other DNA
gaccggacactacccgtac

SEQ ID NO: 3 - DNA, 19 bp, synthetic construct
  source 1..19: mol_type = other DNA
gcttgcaacgtgccaatgc

SEQ ID NO: 4 - DNA, 19 bp, synthetic construct
  source 1..19: mol_type = other DNA
ggccaacatcatggtcgac

SEQ ID NO: 5 - protein, 38 aa, synthetic construct
MDVFMKGLSKAKEGVVAAAEKTKQGVAEAAGKTKEGVL

SEQ ID NO: 6 - protein, 38 aa, synthetic construct
MDVFKKGFSIADEGVVGAVEKTDQGVTEAAEKTKEGVM

SEQ ID NO: 7 - protein, 20 aa, synthetic construct
MDVFKKGFSIADEGVVGAVE

SEQ ID NO: 8 - DNA, 1035 bp, Pseudomonas sp.
  source 1..1035: mol_type = genomic DNA
atgaaactgaaaaacaccttgggcttggccattggttcgcttgtagccgccacttcgattggcgctatggcacaaggtcaaggcgccgtcgagactgaaatcttctacaagaaagaattcttcgacagccagcgcgacttcaagaacgacggcaacctgttcggcggctcgatcggttacttcctgaccgacgacgttgagctgcgtctgggctacgacgaagtgcacaacgctcgtggcgaagacggcaagaacatcaagggctcgaacactgccctggacgccgtttaccacttcaacaacccgtacgacgctatccgtccatacgtttccgctggtttctcgcaccagtcgctgggccagaccggccgtggcggtcgtgaccactccaccttcgccaacgttggcgctggcgccaagtggtacatcaccgacatgttctatgcccgtgccggcgtagaagctcagtacaacatcgaccagggcgacaccgagtgggccccgagcgttggtgttggcctgaacttcggcggtagcccgaagcaagctgaagctgctcctgctccagttgctgaagtctgctccgactccgacaacgacggcgtgtgcgacaacgtcgacaagtgcccagacaccccggccaacgttaccgttgacgccgacggctgcccggctgttgccgaagtcgttcgcgttgagctggacgtcaagttcgacttcgacaagtcggtcgtcaagcctaacagctacggcgacatcaaaaacctggctgacttcatgaagcagtacccacaaaccaccaccgtggttgaaggtcacactgactccgtgggtccagacgcttacaaccagaagctgtccgagcgtcgtgccaacgctgtcaagcaagtgctgacccagcagtacggcgtagaatccagccgtgttgactcggttggctacggcgaaacccgtccggttgctgacaacgccaccgaagaaggccgtgctgtcaaccgtcgcgttgaagctcaggtagaagcccaggccaagtaa

SEQ ID NO: 9 - protein, 344 aa, Pseudomonas sp.
MKLKNTLGLAIGSLVAATSIGAMAQGQGAVETEIFYKKEFFDSQRDFKNDGNLFGGSIGYFLTDDVELRLGYDEVHNARGEDGKNIKGSNTALDAVYHFNNPYDAIRPYVSAGFSHQSLGQTGRGGRDHSTFANVGAGAKWYITDMFYARAGVEAQYNIDQGDTEWAPSVGVGLNFGGSPKQAEAAPAPVAEVCSDSDNDGVCDNVDKCPDTPANVTVDADGCPAVAEVVRVELDVKFDFDKSVVKPNSYGDIKNLADFMKQYPQTTTVVEGHTDSVGPDAYNQKLSERRANAVKQVLTQQYGVESSRVDSVGYGETRPVADNATEEGRAVNRRVEAQVEAQAK

SEQ ID NO: 10 - DNA, 252 bp, Pseudomonas sp.
  source 1..252: mol_type = genomic DNA
atgaacaacgttctgaaattctctgctctggctctggccgcagttctggctaccggttgcagcagcgtatccaaagaaaccgaagctcgtctgactgcgactgaagacgcagcagctcgcgctcaagctcgtgctgacgaagcctaccgtaaggctgacgacgcaatggcagccgctcagaaggctcagcagaccgctgacgaagccaacgagcgcgccctgcgtatgctggacaaagccagccgcaagtaa

SEQ ID NO: 11 - protein, 83 aa, Pseudomonas sp.
MNNVLKFSALALAAVLATGCSSVSKETEARLTATEDAAARAQARADEAYRKADDAMAAAQKAQQTADEANERALRMLDKASRK

SEQ ID NO: 12 - DNA, 924 bp, Pseudomonas sp.
  source 1..924: mol_type = genomic DNA
atgaacaaaggtgtaatgcgaccgggccatgtgcagctgcgtgtactggacatgagcaaggccctggaacactacgtcgagttgctgggcctgatcgagatggaccgtgacgaccagggccgtgtctatctgaaggcttggaccgaagtggataagttttccctggtgctacgcgaggctgacgagccgggcatggattttatgggtttcaaggttgtggatgaggatgctctccggcaactggagcgggatctgatggcatatggctgtgccgttgagcagctacccgcaggtgaactgaacagttgtggccggcgcgtgcgcttccaggccccctccgggcatcacttcgagttgtatgcagacaaggaatatactggaaagtggggtttgaatgacgtcaatcccgaggcatggccgcgcgatctgaaaggtatggcggctgtgcgtttcgaccacgccctcatgtatggcgacgaattgccggcgacctatgacctgttcaccaaggtgctcggtttctatctggccgaacaggtgctggacgaaaatggcacgcgcgtcgcccagtttctcagtctgtcgaccaaggcccacgacgtggccttcattcaccatccggaaaaaggccgcctccatcatgtgtccttccacctcgaaacctgggaagacttgcttcgcgccgccgacctgatctccatgaccgacacatctatcgatatcggcccaacccgccacggcctcactcacggcaagaccatctacttcttcgacccgtccggtaaccgcaacgaagtgttctgcgggggagattacaactacccggaccacaaaccggtgacctggaccaccgaccagctgggcaaggcgatcttttaccacgaccgcattctcaacgaacgattcatgaccgtgctgacctga

SEQ ID NO: 13 - protein, 307 aa, Pseudomonas sp.
MNKGVMRPGHVQLRVLDMSKALEHYVELLGLIEMDRDDQGRVYLKAWTEVDKFSLVLREADEPGMDFMGFKVVDEDALRQLERDLMAYGCAVEQLPAGELNSCGRRVRFQAPSGHHFELYADKEYTGKWGLNDVNPEAWPRDLKGMAAVRFDHALMYGDELPATYDLFTKVLGFYLAEQVLDENGTRVAQFLSLSTKAHDVAFIHHPEKGRLHHVSFHLETWEDLLRAADLISMTDTSIDIGPTRHGLTHGKTIYFFDPSGNRNEVFCGGDYNYPDHKPVTWTTDQLGKAIFYHDRILNERFMTVLT

SEQ ID NO: 14 - DNA, 984 bp, synthetic construct
  source 1..984: mol_type = other DNA
cgtggcgttcctcatattgttatggtggacgcctacaaacgctataaaggcggtggttcgatgaacaaaggtgtaatgcgaccgggccatgtgcagctgcgtgtactggacatgagcaaggccctggaacactacgtcgagttgctgggcctgatcgagatggaccgtgacgaccagggccgtgtctatctgaaggcttggaccgaagtggataagttttccctggtgctacgcgaggctgacgagccgggcatggattttatgggtttcaaggttgtggatgaggatgctctccggcaactggagcgggatctgatggcatatggctgtgccgttgagcagctacccgcaggtgaactgaacagttgtggccggcgcgtgcgcttccaggccccctccgggcatcacttcgagttgtatgcagacaaggaatatactggaaagtggggtttgaatgacgtcaatcccgaggcatggccgcgcgatctgaaaggtatggcggctgtgcgtttcgaccacgccctcatgtatggcgacgaattgccggcgacctatgacctgttcaccaaggtgctcggtttctatctggccgaacaggtgctggacgaaaatggcacgcgcgtcgcccagtttctcagtctgtcgaccaaggcccacgacgtggccttcattcaccatccggaaaaaggccgcctccatcatgtgtccttccacctcgaaacctgggaagacttgcttcgcgccgccgacctgatctccatgaccgacacatctatcgatatcggcccaacccgccacggcctcactcacggcaagaccatctacttcttcgacccgtccggtaaccgcaacgaagtgttctgcgggggagattacaactacccggaccacaaaccggtgacctggaccaccgaccagctgggcaaggcgatcttttaccacgaccgcattctcaacgaacgattcatgaccgtgctgacctga

SEQ ID NO: 15 - protein, 327 aa, synthetic construct
RGVPHIVMVDAYKRYKGGGSMNKGVMRPGHVQLRVLDMSKALEHYVELLGLIEMDRDDQGRVYLKAWTEVDKFSLVLREADEPGMDFMGFKVVDEDALRQLERDLMAYGCAVEQLPAGELNSCGRRVRFQAPSGHHFELYADKEYTGKWGLNDVNPEAWPRDLKGMAAVRFDHALMYGDELPATYDLFTKVLGFYLAEQVLDENGTRVAQFLSLSTKAHDVAFIHHPEKGRLHHVSFHLETWEDLLRAADLISMTDTSIDIGPTRHGLTHGKTIYFFDPSGNRNEVFCGGDYNYPDHKPVTWTTDQLGKAIFYHDRILNERFMTVLT

SEQ ID NO: 16 - DNA, 693 bp, Pseudomonas sp.
  source 1..693: mol_type = genomic DNA
atgagcatagtacgcacagcgttacccctggtactgctcaccagtgtgttgactggttgtgcaggtttgcaaaaaaccgactggccgaaatgtgccgccgtcgggggtgtaggcggcgccgccctgggcgccatcgaaagctccagctgggctggctggggtgcgttgctgggcggtggcctggcggcgggctattgctgggcccatggcgatggcgacgaggatggtgacggcgtgccagacagccgtgacaagtgccctggcaccccgcgtggtgtgcaggtcgatgccaacggatgcccgcctgagccggttgcggtggtcgaagaagtggtggtgcagaaggaagaagtcattgtcatccgcgatgtgcacttcgagttcgattctgcgcgcctgacggccagtgacaaagagcgcctcaataccattgccacgcgcctgaagcaggaagcgccctctgcccgccttagcgtcagcggccataccgacagcgtcggttccgacagctacaaccagaaactgtccgagcgccgtgcccattcggtgaccgattacctggtcgagagcggtgtaccgcgcagcagcttcgtttcggtggtcggcgcgggtgaaacccagccggtggcagacaacgccacggccgaagggcgtgccatgaaccgtcgtaccgagatcaagatccagcggtaa

SEQ ID NO: 17 - protein, 230 aa, Pseudomonas sp.
MSIVRTALPLVLLTSVLTGCAGLQKTDWPKCAAVGGVGGAALGAIESSSWAGWGALLGGGLAAGYCWAHGDGDEDGDGVPDSRDKCPGTPRGVQVDANGCPPEPVAVVEEVVVQKEEVIVIRDVHFEFDSARLTASDKERLNTIATRLKQEAPSARLSVSGHTDSVGSDSYNQKLSERRAHSVTDYLVESGVPRSSFVSVVGAGETQPVADNATAEGRAMNRRTEIKIQR

SEQ ID NO: 18 - DNA, 783 bp, synthetic construct
  source 1..783: mol_type = other DNA
gtgcctcgagcagtggcacgggcgcgtcttcatgccggccagccgtttgtggccaatgacacaggagcattcagcatgagtgtgacgtcgaaggcggctttgccgctgttggtggctgccagcctgctcacgggctgcgctacgcacagcgatggcagcgcgcccctcaatcaaaggacctggcccatctgcagcctgctgggcggcttggtcggtggtggccttggtgccatcgagagttcttcctgggccgccggtggtggcgccttgggcgccattgccggcgggctgatttgttacgcccaggacggtgacgaagatggtgacggcattttcgaccggcgcgatcactgccccgagaccccggccaacacggcggttgaccacatgggctgcccactgaaacagtacccggccgcgccacctgccggtggtggcgggagcgtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactccaattgaatttacagttaatgaggacggtcaggttactgtagatggtgaagcaactgaaggtgacgctcatact

SEQ ID NO: 19 - protein, 261 aa, synthetic construct
VPRAVARARLHAGQPFVANDTGAFSMSVTSKAALPLLVAASLLTGCATHSDGSAPLNQRTWPICSLLGGLVGGGLGAIESSSWAAGGGALGAIAGGLICYAQDGDEDGDGIFDRRDHCPETPANTAVDHMGCPLKQYPAAPPAGGGGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 20 - DNA, 1890 bp, Pseudomonas sp.
  source 1..1890: mol_type = genomic DNA
atgcgaaaagccccgttattgcgctttaccctcgcttcactggccctggcctgcagccaggcgttggccggcccttcgccctattcaaccctgatcgtgtttggcgacagcctcgccgatgccgggcagtttcccgatcttgttggcggtaccccaggcgcgcgtttcaccaaccgtgacgccgacggcaacttcgccccggtgtcgccgatgatcctcggtggccgcctgggcgtcgcgccaggcgaccttaacccgtcgacatccgtaggtatccagcccgatggtaataactgggcagtcggcgggtacaccacccagcagatcctggactcgatcacgacaacgtccgagaccgtcatccccccaggaaaccccaatgccgggttggtgctgcgcgagcgccccggctacctggccaacggcctgcgcgccgaccccaatgccttgtactacctgacaggcggcggcaacgacttccttcagggcctggtgaatagcccggccgacgccgtagccgccggcgcccgcctggctgccagcgcccaagcgcttcagcaaggaggcgcgcgctacatcatggtctggctgctacctgacctcggccaaacgcccaatttcagtggcacgccacagcaaaacccactgtcactgctctccgctgcgttcaaccagtcactgatcagccagctagggcagatcgatgcccagatcattccactgaacatccctttgctgttgagcgaggcgctggccagccccagtcagttcggcctggccagcgaccagaacctggtcggcacctgctatagcggcgatagctgcgtggaaaacccggtgtacgggatcaacggcacaacgccagacccgaccaaactgctgttcaacgactcggtccacccgaccatcgcgggtcagcagctgattgccgattacgcctactcgatcctcgcggccccctgggaactgaccctgctaccggaaatggcccacgccagcctgcgggctcaccaggatgagttgcgtaatcagtggcagacgccttggcaagcagttggccaatggcaagcctttgtcgccagcggcgctcaggacctggacttcgacggccagcacagcgcggccagcggtgacggccgcggctacaacctgaccgtgggcggcagctatcgcctgaacgacgcctggcgcctgggcctggccggcggtgcaaaccggcagaagctggaagctggtgaacaggactcggactacaagctgaacagttatatggccagtgcctttgcccaataccgccaggaccgctggtgggccgacgcggcgctgaccgccgggcacctggattacagcgacctcaagcgtaccttcgccctgggcgtgaatgaccgcagtgagaagggcgacaccgacggcgaggcctgggcaatgtccgggcggctgggctacaacctggcggccgacaccagcaactggcagttggcacctttcatcagtgccgactatgcgcgggtgaaggtggatggctacgacgagaagagcggacgttcgacggcgcttggcttcgatgaccaggagcgcacgtcacgccgcctgggcgtggggctgctgggcagtgtgcaggtactgccaagtacccggcttttcgccgaggtggcgcaggagcatgagttcgaggacgacgagcaggatgtgacgatgcacctgaccagcttgccggcgaatgacttcaccctgaccgggtatacgccgcacagcgacctgacccgggcgagcctgggtgtgagccatgaactggtggcaggggtgcatttgcgcgggaactacaactggcgcaagagtgatgagttgacgcaacagggtattagcgtgggggttagcgtggacttctga

SEQ ID NO: 21 - protein, 629 aa, Pseudomonas sp.
MRKAPLLRFTLASLALACSQALAGPSPYSTLIVFGDSLADAGQFPDLVGGTPGARFTNRDADGNFAPVSPMILGGRLGVAPGDLNPSTSVGIQPDGNNWAVGGYTTQQILDSITTTSETVIPPGNPNAGLVLRERPGYLANGLRADPNALYYLTGGGNDFLQGLVNSPADAVAAGARLAASAQALQQGGARYIMVWLLPDLGQTPNFSGTPQQNPLSLLSAAFNQSLISQLGQIDAQIIPLNIPLLLSEALASPSQFGLASDQNLVGTCYSGDSCVENPVYGINGTTPDPTKLLFNDSVHPTIAGQQLIADYAYSILAAPWELTLLPEMAHASLRAHQDELRNQWQTPWQAVGQWQAFVASGAQDLDFDGQHSAASGDGRGYNLTVGGSYRLNDAWRLGLAGGANRQKLEAGEQDSDYKLNSYMASAFAQYRQDRWWADAALTAGHLDYSDLKRTFALGVNDRSEKGDTDGEAWAMSGRLGYNLAADTSNWQLAPFISADYARVKVDGYDEKSGRSTALGFDDQERTSRRLGVGLLGSVQVLPSTRLFAEVAQEHEFEDDEQDVTMHLTSLPANDFTLTGYTPHSDLTRASLGVSHELVAGVHLRGNYNWRKSDELTQQGISVGVSVDF

SEQ ID NO: 22 - DNA, 1344 bp, synthetic construct
  source 1..1344: mol_type = other DNA
gtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactccaattgaatttacagttaatgaggacggtcaggttactgtagatggtgaagcaactgaaggtgacgctcatactggtggtgggggctccccgaccatcgcgggtcagcagctgattgccgattacgcctactcgatcctcgcggccccctgggaactgaccctgctaccggaaatggcccacgccagcctgcgggctcaccaggatgagttgcgtaatcagtggcagacgccttggcaagcagttggccaatggcaagcctttgtcgccagcggcgctcaggacctggacttcgacggccagcacagcgcggccagcggtgacggccgcggctacaacctgaccgtgggcggcagctatcgcctgaacgacgcctggcgcctgggcctggccggcggtgcaaaccggcagaagctggaagctggtgaacaggactcggactacaagctgaacagttatatggccagtgcctttgcccaataccgccaggaccgctggtgggccgacgcggcgctgaccgccgggcacctggattacagcgacctcaagcgtaccttcgccctgggcgtgaatgaccgcagtgagaagggcgacaccgacggcgaggcctgggcaatgtccgggcggctgggctacaacctggcggccgacaccagcaactggcagttggcacctttcatcagtgccgactatgcgcgggtgaaggtggatggctacgacgagaagagcggacgttcgacggcgcttggcttcgatgaccaggagcgcacgtcacgccgcctgggcgtggggctgctgggcagtgtgcaggtactgccaagtacccggcttttcgccgaggtggcgcaggagcatgagttcgaggacgacgagcaggatgtgacgatgcacctgaccagcttgccggcgaatgacttcaccctgaccgggtatacgccgcacagcgacctgacccgggcgagcctgggtgtgagccatgaactggtggcaggggtgcatttgcgcgggaactacaactggcgcaagagtgatgagttgacgcaacagggtattagcgtgggggttagcgtggacttctga

SEQ ID NO: 23 - protein, 447 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGGGGSPTIAGQQLIADYAYSILAAPWELTLLPEMAHASLRAHQDELRNQWQTPWQAVGQWQAFVASGAQDLDFDGQHSAASGDGRGYNLTVGGSYRLNDAWRLGLAGGANRQKLEAGEQDSDYKLNSYMASAFAQYRQDRWWADAALTAGHLDYSDLKRTFALGVNDRSEKGDTDGEAWAMSGRLGYNLAADTSNWQLAPFISADYARVKVDGYDEKSGRSTALGFDDQERTSRRLGVGLLGSVQVLPSTRLFAEVAQEHEFEDDEQDVTMHLTSLPANDFTLTGYTPHSDLTRASLGVSHELVAGVHLRGNYNWRKSDELTQQGISVGVSVDF

SEQ ID NO: 24 - DNA, 1041 bp, Escherichia coli
  source 1..1041: mol_type = genomic DNA
atgaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggtatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagcagctccagtagttgctccggctccagctccggcaccggaagtacagaccaagcacttcactctgaagtctgacgttctgttcaacttcaacaaagcaaccctgaaaccggaaggtcaggctgctctggatcagctgtacagccagctgagcaacctggatccgaaagacggttccgtagttgttctgggttacaccgaccgcatcggttctgacgcttacaaccagggtctgtccgagcgccgtgctcagtctgttgttgattacctgatctccaaaggtatcccggcagacaagatctccgcacgtggtatgggcgaatccaacccggttactggcaacacctgtgacaacgtgaaacagcgtgctgcactgatcgactgcctggctccggatcgtcgcgtagagatcgaagttaaaggtatcaaagacgttgtaactcagccgcaggcttaa

SEQ ID NO: 25 - protein, 346 aa, Escherichia coli
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 26 - DNA, 930 bp, synthetic construct
  source 1..930: mol_type = other DNA
atgaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggtatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcatgctgagcctgggtgtttcctaccgtttcggtggcggtgggggttcggtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactccaattgaatttacagttaatgaggacggtcaggttactgtagatggtgaagcaactgaaggtgacgctcatact

SEQ ID NO: 27 - protein, 310 aa, synthetic construct
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGGGGGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 28 - DNA, 924 bp, Pseudomonas syringae
  source 1..924: mol_type = genomic DNA
atgactctcgacaaggcgttggtgctgcgtacctgtgcaaataacatggccgatcactgcggccttatatggcccgcgtccggcacggtggaatccagatactggcagtcaaccaggcggcatgagaatggtctggtcggtttactgtggggcgctggaaccagcgcttttctaagcgtgcatgccgatgctcgatggattgtctgtgaagttgccgttgcagacatcatcagtctggaagagccgggaatggtcaagtttccgcgggccgaggtggttcatgtcggcgacaggatcagcgcgtcacacttcatttcggcacgtcaggccgaccctgcgagcaccagcacgtccacgagcacgagcacgttaacgccaatgcctacggccatacccacgcccatgcctgcggtagcaagtgtcacgttaccggtggccgaacaggcccgtcatgaagtgttcgatgtcgcgtcggtcagcgcggctgccgccccagtaaacaccctgccggtgacgacgccgcagaatttgcagaccgccacttacggcagcacgttgagtggcgacaatcacagtcgtctgattgccggttatggcagtaacgagaccgctggcaaccacagtgatctaattggcgggcatgactgcaccctgatggcgggagaccaaagcagattgaccgctggtaagaacagtgtcttgacggcaggcgctcgtagcaaacttattggcagtgaaggctcgacgctctcggctggagaagactccacactaattttcagactctgggacgggaagaggtacaggcaactggtcgccagaacgggtgagaacggtgttgaggccgacataccgtattacgtgaacgaagatgacgatattgtcgataaacccgacgaggacgatgactggatagaggtaaag

SEQ ID NO: 29 - protein, 308 aa, Pseudomonas syringae
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIAGYGSNETAGNHSDLIGGHDCTLMAGDQSRLTAGKNSVLTAGARSKLIGSEGSTLSAGEDSTLIFRLWDGKRYRQLVARTGENGVEADIPYYVNEDDDIVDKPDEDDDWIEVK

SEQ ID NO: 30 - DNA, 1278 bp, synthetic construct
  source 1..1278: mol_type = other DNA
atgactctcgacaaggcgttggtgctgcgtacctgtgcaaataacatggccgatcactgcggccttatatggcccgcgtccggcacggtggaatccagatactggcagtcaaccaggcggcatgagaatggtctggtcggtttactgtggggcgctggaaccagcgcttttctaagcgtgcatgccgatgctcgatggattgtctgtgaagttgccgttgcagacatcatcagtctggaagagccgggaatggtcaagtttccgcgggccgaggtggttcatgtcggcgacaggatcagcgcgtcacacttcatttcggcacgtcaggccgaccctgcgagcaccagcacgtccacgagcacgagcacgttaacgccaatgcctacggccatacccacgcccatgcctgcggtagcaagtgtcacgttaccggtggccgaacaggcccgtcatgaagtgttcgatgtcgcgtcggtcagcgcggctgccgccccagtaaacaccctgccggtgacgacgccgcagaatttgcagaccgccacttacggcagcacgttgagtggcgacaatcacagtcgtctgattgccggttatggcagtaacgagaccgctggcaaccacagtgatctaattggcgggcatgactgcaccctgatggcgggagaccaaagcagattgaccgctggtaagaacagtgtcttgacggcaggcgctcgtagcaaacttattggcagtgaaggctcgacgctctcggctggagaagactccacactaattttcagactctgggacgggaagaggtacaggcaactggtcgccagaacgggtgagaacggtgttgaggccgacataccgtattacgtgaacgaagatgacgatattgtcgataaacccgacgaggacgatgactggatagaggtaaagggcggtgggggttcggtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactccaattgaatttacagttaatgaggacggtcaggttactgtagatggtgaagcaactgaaggtgacgctcatact

SEQ ID NO: 31 - protein, 426 aa, synthetic construct
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIAGYGSNETAGNHSDLIGGHDCTLMAGDQSRLTAGKNSVLTAGARSKLIGSEGSTLSAGEDSTLIFRLWDGKRYRQLVARTGENGVEADIPYYVNEDDDIVDKPDEDDDWIEVKGGGGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 32 - DNA, 792 bp, Pseudomonas sp.
  source 1..792: mol_type = genomic DNA
atgcgcaaacacgtaatgattcccgccctgctggccctgagcgtcggtcttgctgcctgctcgcatgatccgaatgccaacctggaatcggcccgcaccaacttctcctcactgcagagcgacccgcaagcgagcaaagtcgcggcactggagaccaaggacgcccaggactggctgaacaaggccgacaaggcgtacatggaccgtgaagacgagaagaaagtcgaccaactggcctacctgaccaaccagcgcgtcgaagtggccaagcagaccattgccctgcgtactgccgaagctgaactgaaaaacgcctcggcccagcgcgcccaggccaagctggatgcccgcgacgcgcagatcgccaagctgcaggacagcctcaacgccaagcagaccgaccgcggaacgctggtgaccttcggcgacgtgctgttcgacttcaacaaggccgaacttaagagcaacgcctacccgaacatcaccaagctggcccagttccttcaggaaaacccggaacgcaaggtgatcgtcgagggctacaccgacagcgtcggctcggccaactacaaccagaccctgtccgagcgccgtgccaacagcgtgcgcatggcactggtgcgtgccggggtagatccggcgcgtatcgtttcccagggctatggcaaggagtacccggtagcggacaactcgagcaactcgggacgtgcgcagaaccgtcgggtggaggtgaccatctccaacgacaaccagccggtggcaccacgctcggtgagccaggttcagcgctaa

SEQ ID NO: 33 - protein, 263 aa, Pseudomonas sp.
MRKHVMIPALLALSVGLAACSHDPNANLESARTNFSSLQSDPQASKVAALETKDAQDWLNKADKAYMDREDEKKVDQLAYLTNQRVEVAKQTIALRTAEAELKNASAQRAQAKLDARDAQIAKLQDSLNAKQTDRGTLVTFGDVLFDFNKAELKSNAYPNITKLAQFLQENPERKVIVEGYTDSVGSANYNQTLSERRANSVRMALVRAGVDPARIVSQGYGKEYPVADNSSNSGRAQNRRVEVTISNDNQPVAPRSVSQVQR

SEQ ID NO: 34 - DNA, 813 bp, Pseudomonas sp.
  source 1..813: mol_type = genomic DNA
atgatccgtcgtacgcctttggctgcactggcattgctggcgctgacggcaggtttgcaaggttgcgccagtcagcgcagcagtgccgcgctggatgaggccaccgttgccttccagggggtcaaagatgattccgatgtgctgcgcagcgcgccgcgtgacgtgattcgggcgggtgagtcgctggctcgcgccgagcgcctgtccagttacatcggcaccggttccgatgtgcggcattatgcttacctcagccagcgctacagcgagattgcccgcgagcatgccaagctggcgctgaaccaggagcgccaggccaagctcgacctggagcgccagcgcctgcagctggccttgcgtgaggccaaactggccagcgtgcagcagcagggcaagtgggtcgagtcgcagattgccgcgttggcttcggagcaggccgaccgtggcttggtgatgaccttgggcgatgtgctgttcgataccggcagtgccgacctgaagaactcggccagccggactgtgctcaagctggtgcagttcctgcagctcaacccgcgccgggtagtgcgtattgagggctataccgacagtactggcgcgggcgaggagaatctcaagctgtcgcgcgaccgggcgcagtccgtggctgacatgcttgtggacctgggcatcgacgaaaagcgcctgcaggttgaaggctatggcgaccagtacccgatcgaggccaatgcttcggagcggggcagggcgcagaaccgtcgggtggagatcgtattctccgatgacaaggggcggctcgcaccggcgcgctga

SEQ ID NO: 35 - protein, 270 aa, Pseudomonas sp.
MIRRTPLAALALLALTAGLQGCASQRSSAALDEATVAFQGVKDDSDVLRSAPRDVIRAGESLARAERLSSYIGTGSDVRHYAYLSQRYSEIAREHAKLALNQERQAKLDLERQRLQLALREAKLASVQQQGKWVESQIAALASEQADRGLVMTLGDVLFDTGSADLKNSASRTVLKLVQFLQLNPRRVVRIEGYTDSTGAGEENLKLSRDRAQSVADMLVDLGIDEKRLQVEGYGDQYPIEANASERGRAQNRRVEIVFSDDKGRLAPAR

SEQ ID NO: 36 - DNA, 519 bp, Pseudomonas sp.
  source 1..519: mol_type = genomic DNA
atgaaatcaggaacaggagttgaacccgtgatgcacgctttacgttttccgctttgggccttgttgttcgccatgctggcgctgacggggtgccagagcgccccccaaaagggccttaccccagaacagattgccgtgctcaagcgcgaaggtttcaccccgaccgatgaaggttgggcctacgacttgtctggcaaagtgctgttcggcagcgatctggacagcctcaacggccagagccaggcgattgtcgagcgcatcggcaaggcgctgctcggcgtgggtatccagggcgtgcgggtggacgggcatgccgactcgtcgggcaaggcggcgtataaccagcagctgtccgagcgccgcgcgcaaagcgtgaccaaggcgctggtggggattggcatgcaggcacagaacattcagagccgtggcctgggcagcagccagccggtggcggacaaccgcaccagcgccgggcgtaccgagaaccgtcgggtgtccatcgtggtagcgtcctactga

SEQ ID NO: 37 - protein, 172 aa, Pseudomonas sp.
MKSGTGVEPVMHALRFPLWALLFAMLALTGCQSAPQKGLTPEQIAVLKREGFTPTDEGWAYDLSGKVLFGSDLDSLNGQSQAIVERIGKALLGVGIQGVRVDGHADSSGKAAYNQQLSERRAQSVTKALVGIGMQAQNIQSRGLGSSQPVADNRTSAGRTENRRVSIVVASY

SEQ ID NO: 38 - DNA, 705 bp, Branchiostoma lanceolatum
  source 1..705: mol_type = genomic DNA
gtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgcgatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaag

SEQ ID NO: 39 - protein, 235 aa, Branchiostoma lanceolatum
VSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK